(19) United States
(12) Patent Application Publication
(10) Pub. No.: US 2026/0325063 A1
(43) Pub. Date: Oct. 22, 2026

(54) SYSTEMS AND METHODS FOR ANALYZING SOUNDS MADE BY AN INDIVIDUAL DURING A SLEEP SESSION

(71) Applicant: ResMed Digital Health Inc., San Diego, CA (US)

(72) Inventors: Stephen Dodd, Dublin (IE); Graeme Alexander Lyon, Dublin (IE)

(21) Appl. No.: 19/109,851

(22) PCT Filed: Sep. 26, 2023
(86) PCT No.: PCT/IB2023/059559

§ 371 Date: Mar. 7, 2025

Related U.S. Application Data
(60) Provisional application No. 63/410,527, filed on Sep. 27, 2022.

(51) Int. Cl. A61B 5/00

(52) U.S. Cl. CPC A61B 5/4818; A61B 2562/0204

(57) ABSTRACT

A method for analyzing sounds made by an individual during a sleep session includes receiving audio data associated with the sleep session. The sleep session is divided into at least a plurality of primary segments. The method further includes determining, based at least in part on the audio data, whether one or more snoring sounds were made by the individual during each of at least two of the plurality of primary segments. The method further includes determining a snore score for each of a plurality of secondary segments of the sleep session. Each of the plurality of secondary segments contains two or more of the plurality of primary segments. The method further includes generating, based at least in part on the snore score for each of the plurality of secondary segments, a human-perceivable audio sample of at least one of the plurality of secondary segments.

### CROSS-REFERENCE TO RELATED APPLICATIONS

This application claims the benefit of, and priority to, U.S. Provisional Patent Application No. 63/410,527 filed on Sep. 27, 2022, which is hereby incorporated by reference herein in its entirety.

### TECHNICAL FIELD

The present disclosure relates generally to systems and methods for analyzing sounds made by an individual during a sleep session, and more particularly, to systems and methods for determining whether the individual is snoring during the sleep session and for generating audio samples of the individual snoring during the sleep session.

### BACKGROUND

Many individuals suffer from sleep-related and/or respiratory-related disorders such as, for example, Sleep Disordered Breathing (SDB), which can include Obstructive Sleep Apnea (OSA), Central Sleep Apnea (CSA), other types of apneas such as mixed apneas and hypopneas, Respiratory Effort Related Arousal (RERA), and snoring. In some cases, these disorders manifest, or manifest more pronouncedly, when the individual is in a particular lying/sleeping position. These individuals may also suffer from other health conditions (which may be referred to as comorbidities), such as insomnia (e.g., difficulty initiating sleep, frequent or prolonged awakenings after initially falling asleep, and/or an early awakening with an inability to return to sleep), Periodic Limb Movement Disorder (PLMD), Restless Leg Syndrome (RLS), Cheyne-Stokes Respiration (CSR), respiratory insufficiency, Obesity Hyperventilation Syndrome (OHS), Chronic Obstructive Pulmonary Disease (COPD), Neuromuscular Disease (NMD), rapid eye movement (REM) behavior disorder (also referred to as RBD), dream enactment behavior (DEB), hypertension, diabetes, stroke, and chest wall disorders.

These disorders are often preceded or accompanied by the individual snoring during sleep, which can be an indication that the individual should see a healthcare provider and/or undergo treatment. However, individuals are often resistant to the fact that they snore during sleep. Thus, new systems and methods for analyzing sounds made by individuals while they sleep are needed. The present disclosure is directed to solving these and other problems.

### SUMMARY

According to some implementations of the present disclosure, a method for analyzing sounds made by an individual during a sleep session includes receiving audio data associated with the sleep session. The sleep session is divided into at least a plurality of primary segments. The method further includes determining, based at least in part on the audio data, whether one or more snoring sounds were made by the individual during each of at least two of the plurality of primary segments. The method further includes determining a snore score for each of a plurality of secondary segments of the sleep session. Each of the plurality of secondary segments contains two or more of the plurality of primary segments. The method further includes generating, based at least in part on the snore score for each of the plurality of secondary segments, a human-perceivable audio sample of at least one of the plurality of secondary segments.

According to some implementations of the present disclosure, a system for analyzing sounds made by an individual during a sleep session includes an electronic interface configured to receive data, a memory storing machine-readable instructions, and a control system including one or more processors configured to execute the machine-readable instructions to implement a method. The method includes receiving audio data associated with the sleep session. The sleep session being divided into at least a plurality of primary segments. The method further includes determining, based at least in part on the audio data, whether one or more snoring sounds were made by the individual during each of at least two of the plurality of primary segments. The method further includes determining a snore score for each of a plurality of secondary segments of the sleep session. Each of the plurality of secondary segments contains two or more of the plurality of primary segments. The method further includes generating, based at least in part on the snore score for each of the plurality of secondary segments, a human-perceivable audio sample of at least one of the plurality of secondary segments.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a system, according to some implementations of the present disclosure;

FIG. 2 is a perspective view of at least a portion of the system of FIG. 1, a user, and a bed partner, according to some implementations of the present disclosure;

FIG. 3 illustrates an exemplary timeline for a sleep session, according to some implementations of the present disclosure;

FIG. 4 illustrates an exemplary hypnogram associated with the sleep session of FIG. 3, according to some implementations of the present disclosure;

FIG. 5 is a flow diagram of a method for analyzing sounds made by an individual during a sleep session, according to some implementations of the present disclosure;

FIG. 6A illustrates a sleep session being divided into a plurality of primary segments and a plurality of secondary segments, according to some implementations of the present disclosure; and

FIG. 6B illustrates a sleep session being divided into a plurality of primary segments and a plurality of tertiary segments, according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

The present disclosure is described with reference to the attached figures, where like reference numerals are used throughout the figures to designate similar or equivalent elements. The figures are not drawn to scale, and are provided merely to illustrate the instant disclosure. Several aspects of the disclosure are described below with reference to example applications for illustration.

Many individuals suffer from sleep-related and/or respiratory disorders, such as Sleep Disordered Breathing (SDB) such as Obstructive Sleep Apnea (OSA), Central Sleep Apnea (CSA) and other types of apneas, Respiratory Effort Related Arousal (RERA), snoring, Cheyne-Stokes Respiration (CSR), respiratory insufficiency, Obesity Hyperventilation Syndrome (OHS), Chronic Obstructive Pulmonary Disease (COPD), Periodic Limb Movement Disorder (PLMD), Restless Leg Syndrome (RLS), Neuromuscular Disease (NMD), and chest wall disorders.

Obstructive Sleep Apnea (OSA), a form of Sleep Disordered Breathing (SDB), is characterized by events including occlusion or obstruction of the upper air passage during sleep resulting from a combination of an abnormally small upper airway and the normal loss of muscle tone in the region of the tongue, soft palate and posterior oropharyngeal wall. More generally, an apnea generally refers to the cessation of breathing caused by blockage of the air (Obstructive Sleep Apnea) or the stopping of the breathing function (often referred to as Central Sleep Apnea). CSA results when the brain temporarily stops sending signals to the muscles that control breathing. Typically, the individual will stop breathing for between about 15 seconds and about 30 seconds during an obstructive sleep apnea event.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

A Respiratory Effort Related Arousal (RERA) event is typically characterized by an increased respiratory effort for ten seconds or longer leading to arousal from sleep and which does not fulfill the criteria for an apnea or hypopnea event. RERAs are defined as a sequence of breaths characterized by increasing respiratory effort leading to an arousal from sleep, but which does not meet criteria for an apnea or hypopnea. These events fulfil the following criteria: (1) a pattern of progressively more negative esophageal pressure, terminated by a sudden change in pressure to a less negative level and an arousal, and (2) the event lasts ten seconds or longer. In some implementations, a Nasal Cannula/Pressure Transducer System is adequate and reliable in the detection of RERAs. A RERA detector may be based on a real flow signal derived from a respiratory therapy device. For example, a flow limitation measure may be determined based on a flow signal. A measure of arousal may then be derived as a function of the flow limitation measure and a measure of sudden increase in ventilation. One such method is described in WO 2008/138040 and U.S. Pat. No. 9,358,353, assigned to ResMed Ltd., the disclosure of each of which is hereby incorporated by reference herein in their entireties.

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung. COPD encompasses a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea. As will be understood, a sleep session as described herein can alternatively be referred to as a therapy session, during which an individual may receive respiratory therapy, or can comprise or consist of a therapy session.

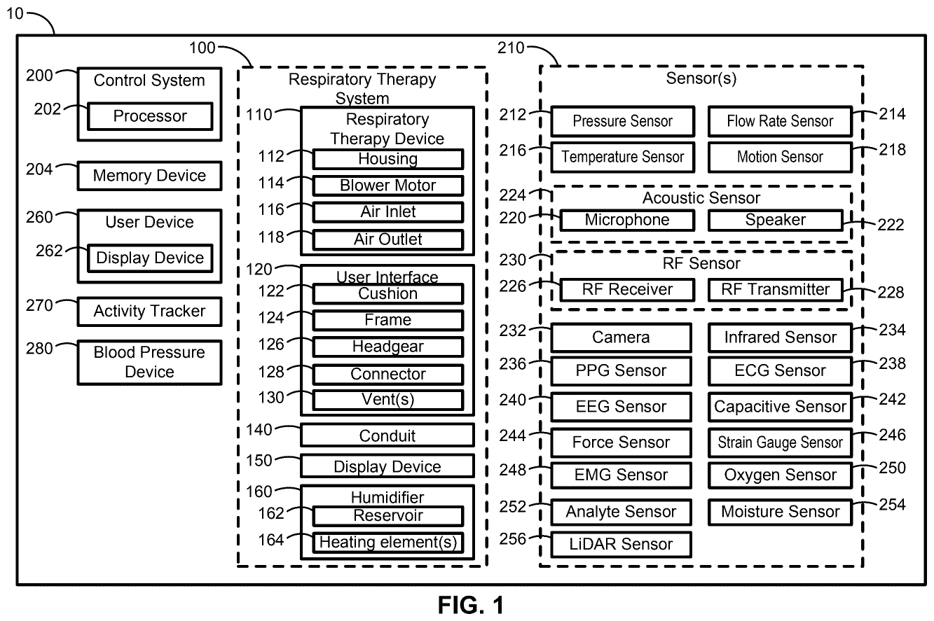

Referring to FIG. 1, a system 10, according to some implementations of the present disclosure, is illustrated. The system 10 can include a respiratory therapy system 100, a control system 200, a memory device 204, and one or more sensors 210. The system 10 may additionally or alternatively include a user device 260, an activity tracker 270, and a blood pressure device 280. The system 10 can be used to analyze data (such as audio data) that is associated with a sleep session of an individual to determine whether the individual snores (e.g., makes one or more snoring sounds) during the sleep session.

The respiratory therapy system 100 includes a respiratory pressure therapy (RPT) device 110 (referred to herein as respiratory therapy device 110), a user interface 120 (also referred to as a mask or a patient interface), a conduit 140 (also referred to as a tube or an air circuit), a display device 150, and a humidifier 160. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy system 100 is generally used to treat individuals suffering from one or more sleep-related respiratory disorders (e.g., obstructive sleep apnea, central sleep apnea, or mixed sleep apnea).

The respiratory therapy system 100 can be used, for example, as a ventilator or as a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

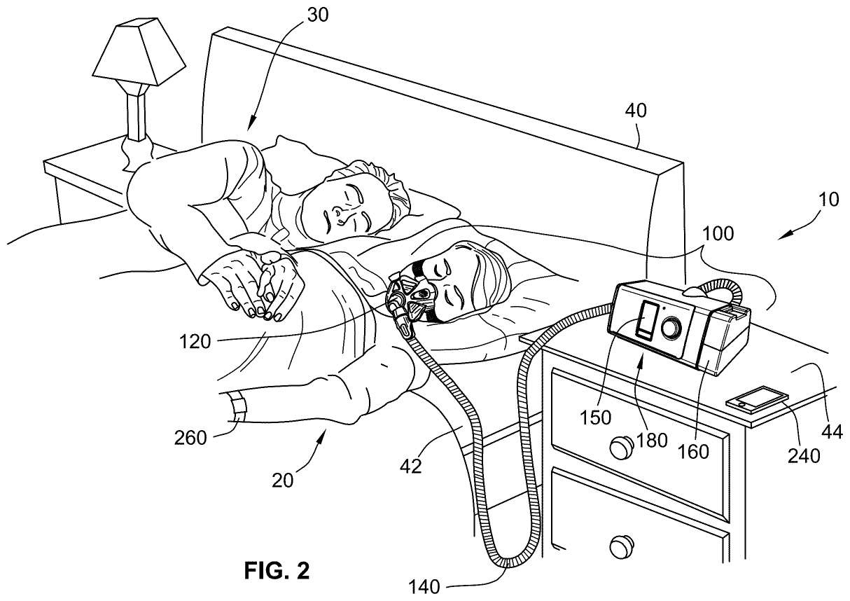

As shown in FIG. 2, the respiratory therapy system 100 can be used to treat a user 20. In this example, the user 20 of the respiratory therapy system 100 and a bed partner 30 are located in a bed 40 and are laying on a mattress 42. The user interface 120 can be worn by the user 20 during a sleep session. The respiratory therapy system 100 generally aids in increasing the air pressure in the throat of the user 20 to aid in preventing the airway from closing and/or narrowing during sleep. The respiratory therapy device 110 can be positioned on a nightstand 44 that is directly adjacent to the bed 40 as shown in FIG. 2, or more generally, on any surface or structure that is generally adjacent to the bed 40 and/or the user 20.

Referring back to FIG. 1, the respiratory therapy device 110 is generally used to generate pressurized air that is delivered to a user (e.g., using one or more motors that drive one or more compressors). In some implementations, the respiratory therapy device 110 generates continuous constant air pressure that is delivered to the user. In other implementations, the respiratory therapy device 110 generates two or more predetermined pressures (e.g., a first predetermined air pressure and a second predetermined air pressure). In still other implementations, the respiratory therapy device 110 generates a variety of different air pressures within a predetermined range. For example, the respiratory therapy device 110 can deliver at least about 6 cmH₂O, at least about 10 cmH₂O, at least about 20 cmH₂O, between about 6 cmH₂O and about 10 cmH₂O, between about 7 cmH₂O and about 12 cmH₂O, etc. The respiratory therapy device 110 can also deliver pressurized air at a predetermined flow rate between, for example, about −20 L/min and about 150 L/min, while maintaining a positive pressure (relative to the ambient pressure).

The respiratory therapy device 110 includes a housing 112, a blower motor 114, an air inlet 116, and an air outlet 118. The blower motor 114 is at least partially disposed or integrated within the housing 112. The blower motor 114 draws air from outside the housing 112 (e.g., atmosphere) via the air inlet 116 and causes pressurized air to flow through the humidifier 160, and through the air outlet 118. In some implementations, the air inlet 116 and/or the air outlet 118 include a cover that is moveable between a closed position and an open position (e.g., to prevent or inhibit air from flowing through the air inlet 116 or the air outlet 118). The housing 112 can also include a vent to allow air to pass through the housing 112 to the air inlet 116. As described below, the conduit 140 is coupled to the air outlet 118 of the respiratory therapy device 110.

The user interface 120 engages a portion of the user's face and delivers pressurized air from the respiratory therapy device 110 to the user's airway to aid in preventing the airway from narrowing and/or collapsing during sleep. This may also increase the user's oxygen intake during sleep. Generally, the user interface 120 engages the user's face such that the pressurized air is delivered to the user's airway via the user's mouth, the user's nose, or both the user's mouth and nose. Together, the respiratory therapy device 110, the user interface 120, and the conduit 140 form an air pathway fluidly coupled with an airway of the user. The pressurized air also increases the user's oxygen intake during sleep. Depending upon the therapy to be applied, the user interface 120 may form a seal, for example, with a region or portion of the user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cmH₂O.

The user interface 120 can include, for example, a cushion 122, a frame 124, a headgear 126, connector 128, and one or more vents 130. The cushion 122 and the frame 124 define a volume of space around the mouth and/or nose of the user. When the respiratory therapy system 100 is in use, this volume space receives pressurized air (e.g., from the respiratory therapy device 110 via the conduit 140) for passage into the airway(s) of the user. The headgear 126 is generally used to aid in positioning and/or stabilizing the user interface 120 on a portion of the user (e.g., the face), and along with the cushion 122 (which, for example, can comprise silicone, plastic, foam, etc.) aids in providing a substantially air-tight seal between the user interface 120 and the user 20. In some implementations the headgear 126 includes one or more straps (e.g., including hook and loop fasteners). The connector 128 is generally used to couple (e.g., connect and fluidly couple) the conduit 140 to the cushion 122 and/or frame 124. Alternatively, the conduit 140 can be directly coupled to the cushion 122 and/or frame 124 without the connector 128. The one or more vents 130 can be used for permitting the escape of carbon dioxide and other gases exhaled by the user 20. The user interface 120 generally can include any suitable number of vents (e.g., one, two, five, ten, etc.).

As shown in FIG. 2, in some implementations, the user interface 120 is a facial mask (e.g., a full face mask) that covers at least a portion of the nose and mouth of the user 20. Alternatively, the user interface 120 can be a nasal mask that provides air to the nose of the user or a nasal pillow mask that delivers air directly to the nostrils of the user 20. In other implementations, the user interface 120 includes a mouthpiece (e.g., a night guard mouthpiece molded to conform to the teeth of the user, a mandibular repositioning device, etc.).

Referring back to FIG. 1, the conduit 140 (also referred to as an air circuit or tube) allows the flow of air between components of the respiratory therapy system 100, such as between the respiratory therapy device 110 and the user interface 120. In some implementations, there can be separate limbs of the conduit for inhalation and exhalation. In other implementations, a single limb conduit is used for both inhalation and exhalation.

The conduit 140 includes a first end that is coupled to the air outlet 118 of the respiratory therapy device 110. The first end can be coupled to the air outlet 118 of the respiratory therapy device 110 using a variety of techniques (e.g., a press fit connection, a snap fit connection, a threaded connection, etc.). In some implementations, the conduit 140 includes one or more heating elements that heat the pressurized air flowing through the conduit 140 (e.g., heat the air to a predetermined temperature or within a range of predetermined temperatures). Such heating elements can be coupled to and/or imbedded in the conduit 140. In such implementations, the first end can include an electrical contact that is electrically coupled to the respiratory therapy device 110 to power the one or more heating elements of the conduit 140. For example, the electrical contact can be electrically coupled to an electrical contact of the air outlet 118 of the respiratory therapy device 110. In this example, electrical contact of the conduit 140 can be a male connector and the electrical contact of the air outlet 118 can be female connector, or, alternatively, the opposite configuration can be used.

The display device 150 is generally used to display image(s) including still images, video images, or both and/or information regarding the respiratory therapy device 110. For example, the display device 150 can provide information regarding the status of the respiratory therapy device 110 (e.g., whether the respiratory therapy device 110 is on/off, the pressure of the air being delivered by the respiratory therapy device 110, the temperature of the air being delivered by the respiratory therapy device 110, etc.) and/or other information (e.g., a sleep score and/or a therapy score, also referred to as a myAir™ score, such as described in WO 2016/061629 and U.S. Patent Pub. No. 2017/0311879, which are hereby incorporated by reference herein in their entireties, the current date/time, personal information for the user 20, etc.). In some implementations, the display device 150 acts as a human-machine interface (HMI) that includes a graphic user interface (GUI) configured to display the image(s) as an input interface. The display device 150 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory therapy device 110.

The humidifier 160 is coupled to or integrated in the respiratory therapy device 110 and includes a reservoir 162 for storing water that can be used to humidify the pressurized air delivered from the respiratory therapy device 110. The humidifier 160 includes a one or more heating elements 164 to heat the water in the reservoir to generate water vapor. The humidifier 160 can be fluidly coupled to a water vapor inlet of the air pathway between the blower motor 114 and the air outlet 118, or can be formed in-line with the air pathway between the blower motor 114 and the air outlet 118. For example, air flows from the air inlet 116 through the blower motor 114, and then through the humidifier 160 before exiting the respiratory therapy device 110 via the air outlet 118.

While the respiratory therapy system 100 has been described herein as including each of the respiratory therapy device 110, the user interface 120, the conduit 140, the display device 150, and the humidifier 160, more or fewer components can be included in a respiratory therapy system according to implementations of the present disclosure. For example, a first alternative respiratory therapy system includes the respiratory therapy device 110, the user interface 120, and the conduit 140. As another example, a second alternative system includes the respiratory therapy device 110, the user interface 120, and the conduit 140, and the display device 150. Thus, various respiratory therapy systems can be formed using any portion or portions of the components shown and described herein and/or in combination with one or more other components.

The control system 200 includes one or more processors 202 (hereinafter, processor 202). The control system 200 is generally used to control (e.g., actuate) the various components of the system 10 and/or analyze data obtained and/or generated by the components of the system 10. The processor 202 can be a general or special purpose processor or microprocessor. While one processor 202 is illustrated in FIG. 1, the control system 200 can include any number of processors (e.g., one processor, two processors, five processors, ten processors, etc.) that can be in a single housing, or located remotely from each other. The control system 200 (or any other control system) or a portion of the control system 200 such as the processor 202 (or any other processor(s) or portion(s) of any other control system), can be used to carry out one or more steps of any of the methods described and/or claimed herein. The control system 200 can be coupled to and/or positioned within, for example, a housing of the user device 260, a portion (e.g., the respiratory therapy device 110) of the respiratory therapy system 100, and/or within a housing of one or more of the sensors 210. The control system 200 can be centralized (within one such housing) or decentralized (within two or more of such housings, which are physically distinct). In such implementations including two or more housings containing the control system 200, the housings can be located proximately and/or remotely from each other.

The memory device 204 stores machine-readable instructions that are executable by the processor 202 of the control system 200. The memory device 204 can be any suitable computer readable storage device or media, such as, for example, a random or serial access memory device, a hard drive, a solid state drive, a flash memory device, etc. While one memory device 204 is shown in FIG. 1, the system 10 can include any suitable number of memory devices 204 (e.g., one memory device, two memory devices, five memory devices, ten memory devices, etc.). The memory device 204 can be coupled to and/or positioned within a housing of a respiratory therapy device 110 of the respiratory therapy system 100, within a housing of the user device 260, within a housing of one or more of the sensors 210, or any combination thereof. Like the control system 200, the memory device 204 can be centralized (within one such housing) or decentralized (within two or more of such housings, which are physically distinct). Thus, while the control system 200 and the memory device 204 are shown as independent components in the block diagram of FIG. 1, they may be components of some other component of the system 10, such as the user device 260, the respiratory therapy device 110, etc.

In some implementations, the memory device 204 stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a geographic location of the user, a relationship status, a family history of insomnia or sleep apnea, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

As described herein, the processor 202 and/or memory device 204 can receive data (e.g., physiological data and/or audio data) from the one or more sensors 210 such that the data for storage in the memory device 204 and/or for analysis by the processor 202. The processor 202 and/or memory device 204 can communicate with the one or more sensors 210 using a wired connection or a wireless connection (e.g., using an RF communication protocol, a Wi-Fi communication protocol, a Bluetooth communication protocol, over a cellular network, etc.). In some implementations, the system 10 can include an antenna, a receiver (e.g., an RF receiver), a transmitter (e.g., an RF transmitter), a transceiver, or any combination thereof. Such components can be coupled to or integrated a housing of the control system 200 (e.g., in the same housing as the processor 202 and/or memory device 204), or the user device 260.

The one or more sensors 210 include a pressure sensor 212, a flow rate sensor 214, temperature sensor 216, a motion sensor 218, a microphone 220, a speaker 222, a radio-frequency (RF) receiver 226, a RF transmitter 228, a camera 232, an infrared (IR) sensor 234, a photoplethysmogram (PPG) sensor 236, an electrocardiogram (ECG) sensor 238, an electroencephalography (EEG) sensor 240, a capacitive sensor 242, a force sensor 244, a strain gauge sensor 246, an electromyography (EMG) sensor 248, an oxygen sensor 250, an analyte sensor 252, a moisture sensor 254, a Light Detection and Ranging (LiDAR) sensor 256, or any combination thereof. Generally, each of the one or more sensors 210 are configured to output sensor data that is received and stored in the memory device 204 or one or more other memory devices.

While the one or more sensors 210 are shown and described as including each of the pressure sensor 212, the flow rate sensor 214, the temperature sensor 216, the motion sensor 218, the microphone 220, the speaker 222, the RF receiver 226, the RF transmitter 228, the camera 232, the IR sensor 234, the PPG sensor 236, the ECG sensor 238, the EEG sensor 240, the capacitive sensor 242, the force sensor 244, the strain gauge sensor 246, the EMG sensor 248, the oxygen sensor 250, the analyte sensor 252, the moisture sensor 254, and the LiDAR sensor 256, more generally, the one or more sensors 210 can include any combination and any number of each of the sensors described and/or shown herein.

As described herein, the system 10 generally can be used to generate physiological data associated with a user (e.g., a user of the respiratory therapy system 100) during a sleep session. The physiological data can be analyzed to generate one or more sleep-related parameters, which can include any parameter, measurement, etc. related to the user during the sleep session. The one or more sleep-related parameters that can be determined for the user 20 during the sleep session include, for example, an Apnea-Hypopnea Index (AHI) score, a sleep score, a flow signal, a respiration signal, a respiration rate, an inspiration amplitude, an expiration amplitude, an inspiration-expiration ratio, a number of events per hour, a pattern of events, a stage, pressure settings of the respiratory therapy device 110, a heart rate, a heart rate variability, movement of the user 20, temperature, EEG activity, EMG activity, arousal, snoring, choking, coughing, whistling, wheezing, or any combination thereof.

The one or more sensors 210 can be used to generate, for example, physiological data, audio data, or both. Physiological data generated by one or more of the sensors 210 can be used by the control system 200 to determine a sleep-wake signal associated with the user 20 during the sleep session and one or more sleep-related parameters. The sleep-wake signal can be indicative of one or more sleep states, including wakefulness, relaxed wakefulness, micro-awakenings, or distinct sleep stages such as, for example, a rapid eye movement (REM) stage, a first non-REM stage (often referred to as “N1”), a second non-REM stage (often referred to as “N2”), a third non-REM stage (often referred to as “N3”), or any combination thereof. Methods for determining sleep states and/or sleep stages from physiological data generated by one or more sensors, such as the one or more sensors 210, are described in, for example, WO 2014/047310, U.S. Patent Pub. No. 2014/0088373, WO 2017/132726, WO 2019/122413, WO 2019/122414, and U.S. Patent Pub. No. 2020/0383580 each of which is hereby incorporated by reference herein in its entirety.

In some implementations, the sleep-wake signal described herein can be timestamped to indicate a time that the user enters the bed, a time that the user exits the bed, a time that the user attempts to fall asleep, etc. The sleep-wake signal can be measured by the one or more sensors 210 during the sleep session at a predetermined sampling rate, such as, for example, one sample per second, one sample per 30 seconds, one sample per minute, etc. In some implementations, the sleep-wake signal can also be indicative of a respiration signal, a respiration rate, an inspiration amplitude, an expiration amplitude, an inspiration-expiration ratio, a number of events per hour, a pattern of events, pressure settings of the respiratory therapy device 110, or any combination thereof during the sleep session. The event(s) can include snoring, apneas, central apneas, obstructive apneas, mixed apneas, hypopneas, a mask leak (e.g., from the user interface 120), a restless leg, a sleeping disorder, choking, an increased heart rate, labored breathing, an asthma attack, an epileptic episode, a seizure, or any combination thereof. The one or more sleep-related parameters that can be determined for the user during the sleep session based on the sleep-wake signal include, for example, a total time in bed, a total sleep time, a sleep onset latency, a wake-after-sleep-onset parameter, a sleep efficiency, a fragmentation index, or any combination thereof. As described in further detail herein, the physiological data and/or the sleep-related parameters can be analyzed to determine one or more sleep-related scores.

Physiological data and/or audio data generated by the one or more sensors 210 can also be used to determine a respiration signal associated with a user during a sleep session. The respiration signal is generally indicative of respiration or breathing of the user during the sleep session. The respiration signal can be indicative of and/or analyzed to determine (e.g., using the control system 200) one or more sleep-related parameters, such as, for example, a respiration rate, a respiration rate variability, an inspiration amplitude, an expiration amplitude, an inspiration-expiration ratio, an occurrence of one or more events, a number of events per hour, a pattern of events, a sleep state, a sleet stage, an apnea-hypopnea index (AHI), pressure settings of the respiratory therapy device 110, or any combination thereof. The one or more events can include snoring, apneas, central apneas, obstructive apneas, mixed apneas, hypopneas, a mask leak (e.g., from the user interface 120), a cough, a restless leg, a sleeping disorder, choking, an increased heart rate, labored breathing, an asthma attack, an epileptic episode, a seizure, increased blood pressure, or any combination thereof. Many of the described sleep-related parameters are physiological parameters, although some of the sleep-related parameters can be considered to be non-physiological parameters. Other types of physiological and/or non-physiological parameters can also be determined, either from the data from the one or more sensors 210, or from other types of data.

The pressure sensor 212 outputs pressure data that can be stored in the memory device 204 and/or analyzed by the processor 202 of the control system 200. In some implementations, the pressure sensor 212 is an air pressure sensor (e.g., barometric pressure sensor) that generates sensor data indicative of the respiration (e.g., inhaling and/or exhaling) of the user of the respiratory therapy system 100 and/or ambient pressure. In such implementations, the pressure sensor 212 can be coupled to or integrated in the respiratory therapy device 110. The pressure sensor 212 can be, for example, a capacitive sensor, an electromagnetic sensor, a piezoelectric sensor, a strain-gauge sensor, an optical sensor, a potentiometric sensor, or any combination thereof.

The flow rate sensor 214 outputs flow rate data that can be stored in the memory device 204 and/or analyzed by the processor 202 of the control system 200. Examples of flow rate sensors (such as, for example, the flow rate sensor 214) are described in International Publication No. WO 2012/012835 and U.S. Pat. No. 10,328,219, both of which are hereby incorporated by reference herein in their entireties. In some implementations, the flow rate sensor 214 is used to determine an air flow rate from the respiratory therapy device 110, an air flow rate through the conduit 140, an air flow rate through the user interface 120, or any combination thereof. In such implementations, the flow rate sensor 214 can be coupled to or integrated in the respiratory therapy device 110, the user interface 120, or the conduit 140. The flow rate sensor 214 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof. In some implementations, the flow rate sensor 214 is configured to measure a vent flow (e.g., intentional “leak”), an unintentional leak (e.g., mouth leak and/or mask leak), a patient flow (e.g., air into and/or out of lungs), or any combination thereof. In some implementations, the flow rate data can be analyzed to determine cardiogenic oscillations of the user. In some examples, the pressure sensor 212 can be used to determine a blood pressure of a user.

The temperature sensor 216 outputs temperature data that can be stored in the memory device 204 and/or analyzed by the processor 202 of the control system 200. In some implementations, the temperature sensor 216 generates temperatures data indicative of a core body temperature of the user 20, a skin temperature of the user 20, a temperature of the air flowing from the respiratory therapy device 110 and/or through the conduit 140, a temperature in the user interface 120, an ambient temperature, or any combination thereof. The temperature sensor 216 can be, for example, a thermocouple sensor, a thermistor sensor, a silicon band gap temperature sensor or semiconductor-based sensor, a resistance temperature detector, or any combination thereof.

The motion sensor 218 outputs motion data that can be stored in the memory device 204 and/or analyzed by the processor 202 of the control system 200. The motion sensor 218 can be used to detect movement of the user 20 during the sleep session, and/or detect movement of any of the components of the respiratory therapy system 100, such as the respiratory therapy device 110, the user interface 120, or the conduit 140. The motion sensor 218 can include one or more inertial sensors, such as accelerometers, gyroscopes, and magnetometers. In some implementations, the motion sensor 218 can comprise an acoustic sensor (such as the acoustic sensor 224 discussed herein) and/or an RF sensor (such as the RF sensor 230 discussed herein), which can generate motion data as further discussed herein. In such implementations, the motion sensor 218, the acoustic sensor, and/or the RF sensor can be disposed in a portable device, such as the user device 260 or the portable device 550 discussed herein. Further, while FIG. 1 and FIG. 2 show the respiratory therapy device 110 as including its own display device 150, in some implementations the respiratory therapy device 110 may not include its own display device, as is discussed herein. In some implementations, the motion sensor 218 alternatively or additionally generates one or more signals representing bodily movement of the user, from which may be obtained a signal representing a sleep state of the user, for example, via a respiratory movement of the user. In some implementations, the motion data from the motion sensor 218 can be used in conjunction with additional data from another one of the sensors 210 to determine the sleep state of the user.

The microphone 220 outputs sound and/or audio data that can be stored in the memory device 204 and/or analyzed by the processor 202 of the control system 200. The audio data generated by the microphone 220 is reproducible as one or more sound(s) during a sleep session (e.g., sounds from the user 20). The audio data form the microphone 220 can also be used to identify (e.g., using the control system 200) an event experienced by the user during the sleep session, as described in further detail herein. The microphone 220 can be coupled to or integrated in the respiratory therapy device 110, the user interface 120, the conduit 140, or the user device 260. The microphone 220 can be coupled to or integrated in a wearable device, such as a smartwatch, smart glasses, earphones or earbuds, or other head-wearable devices. In some implementations, the system 10 includes a plurality of microphones (e.g., two or more microphones and/or an array of microphones with beamforming) such that sound data generated by each of the plurality of microphones can be used to discriminate the sound data generated by another of the plurality of microphones.

The speaker 222 outputs sound waves that are audible to a user of the system 10 (e.g., the user 20 of FIG. 2). The speaker 222 can be used, for example, as an alarm clock or to play an alert or message to the user 20 (e.g., in response to an event). In some implementations, the speaker 222 can be used to communicate the audio data generated by the microphone 220 to the user. The speaker 222 can be coupled to or integrated in the respiratory therapy device 110, the user interface 120, the conduit 140, or the user device 260, and/or can be coupled to or integrated in a wearable device, such as a smartwatch, smart glasses, earphones or ear buds, or other head-wearable devices.

The microphone 220 and the speaker 222 can be used as separate devices. In some implementations, the microphone 220 and the speaker 222 can be combined into an acoustic sensor 224 (e.g., a sonar sensor), as described in, for example, WO 2018/050913, WO 2020/104465, U.S. Pat. App. Pub. No. 2022/0007965, each of which is hereby incorporated by reference herein in its entirety. In such implementations, the speaker 222 generates or emits sound waves at a predetermined interval and the microphone 220 detects the reflections of the emitted sound waves from the speaker 222. The sound waves generated or emitted by the speaker 222 have a frequency that is not audible to the human ear (e.g., below 20 Hz or above around 18 kHz) so as not to disturb the sleep of the user 20 or the bed partner 30. Based at least in part on the data from the microphone 220 and/or the speaker 222, the control system 200 can determine a location of the user 20 and/or one or more of the sleep-related parameters described in herein such as, for example, a respiration signal, a respiration rate, an inspiration amplitude, an expiration amplitude, an inspiration-expiration ratio, a number of events per hour, a pattern of events, a sleep state, a sleep stage, pressure settings of the respiratory therapy device 110, or any combination thereof. In such a context, a sonar sensor may be understood to concern an active acoustic sensing, such as by generating and/or transmitting ultrasound and/or low frequency ultrasound sensing signals (e.g., in a frequency range of about 17-23 kHz, 18-22 kHz, or 17-18 kHz, for example), through the air.

In some implementations, the sensors 210 include (i) a first microphone that is the same as, or similar to, the microphone 220, and is integrated in the acoustic sensor 224 and (ii) a second microphone that is the same as, or similar to, the microphone 220, but is separate and distinct from the first microphone that is integrated in the acoustic sensor 224.

The RF transmitter 228 generates and/or emits radio waves having a predetermined frequency and/or a predetermined amplitude (e.g., within a high frequency band, within a low frequency band, long wave signals, short wave signals, etc.). The RF receiver 226 detects the reflections of the radio waves emitted from the RF transmitter 228, and this data can be analyzed by the control system 200 to determine a location of the user and/or one or more of the sleep-related parameters described herein. An RF receiver (either the RF receiver 226 and the RF transmitter 228 or another RF pair) can also be used for wireless communication between the control system 200, the respiratory therapy device 110, the one or more sensors 210, the user device 260, or any combination thereof. While the RF receiver 226 and RF transmitter 228 are shown as being separate and distinct elements in FIG. 1, in some implementations, the RF receiver 226 and RF transmitter 228 are combined as a part of an RF sensor 230 (e.g., a radar sensor). In some such implementations, the RF sensor 230 includes a control circuit. The format of the RF communication can be Wi-Fi, Bluetooth, or the like.

In some implementations, the RF sensor 230 is a part of a mesh system. One example of a mesh system is a Wi-Fi mesh system, which can include mesh nodes, mesh router(s), and mesh gateway(s), each of which can be mobile/movable or fixed. In such implementations, the Wi-Fi mesh system includes a Wi-Fi router and/or a Wi-Fi controller and one or more satellites (e.g., access points), each of which include an RF sensor that the is the same as, or similar to, the RF sensor 230. The Wi-Fi router and satellites continuously communicate with one another using Wi-Fi signals. The Wi-Fi mesh system can be used to generate motion data based on changes in the Wi-Fi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 232 outputs image data reproducible as one or more images (e.g., still images, video images, thermal images, or any combination thereof) that can be stored in the memory device 204. The image data from the camera 232 can be used by the control system 200 to determine one or more of the sleep-related parameters described herein, such as, for example, one or more events (e.g., periodic limb movement or restless leg syndrome), a respiration signal, a respiration rate, an inspiration amplitude, an expiration amplitude, an inspiration-expiration ratio, a number of events per hour, a pattern of events, a sleep state, a sleep stage, or any combination thereof. Further, the image data from the camera 232 can be used to, for example, identify a location of the user, to determine chest movement of the user, to determine air flow of the mouth and/or nose of the user, to determine a time when the user enters the bed, and to determine a time when the user exits the bed. In some implementations, the camera 232 includes a wide angle lens or a fisheye lens.

The IR sensor 234 outputs infrared image data reproducible as one or more infrared images (e.g., still images, video images, or both) that can be stored in the memory device 204. The infrared data from the IR sensor 234 can be used to determine one or more sleep-related parameters during a sleep session, including a temperature of the user 20 and/or movement of the user 20. The IR sensor 234 can also be used in conjunction with the camera 232 when measuring the presence, location, and/or movement of the user 20. The IR sensor 234 can detect infrared light having a wavelength between about 700 nm and about 1 mm, for example, while the camera 232 can detect visible light having a wavelength between about 380 nm and about 740 nm.

The PPG sensor 236 outputs physiological data associated with the user 20 that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 236 can be worn by the user 20, embedded in clothing and/or fabric that is worn by the user 20, embedded in and/or coupled to the user interface 120 and/or its associated headgear (e.g., straps, etc.), etc.

The ECG sensor 238 outputs physiological data associated with electrical activity of the heart of the user 20. In some implementations, the ECG sensor 238 includes one or more electrodes that are positioned on or around a portion of the user 20 during the sleep session. The physiological data from the ECG sensor 238 can be used, for example, to determine one or more of the sleep-related parameters described herein.

The EEG sensor 240 outputs physiological data associated with electrical activity of the brain of the user 20. In some implementations, the EEG sensor 240 includes one or more electrodes that are positioned on or around the scalp of the user 20 during the sleep session. The physiological data from the EEG sensor 240 can be used, for example, to determine a sleep state and/or a sleep stage of the user 20 at any given time during the sleep session. In some implementations, the EEG sensor 240 can be integrated in the user interface 120, into associated headgear (e.g., straps, etc.), into a head band or other head-worn sensor device, etc.

The capacitive sensor 242, the force sensor 244, and the strain gauge sensor 246 output data that can be stored in the memory device 204 and used/analyzed by the control system 200 to determine, for example, one or more of the sleep-related parameters described herein. The EMG sensor 248 outputs physiological data associated with electrical activity produced by one or more muscles. The oxygen sensor 250 outputs oxygen data indicative of an oxygen concentration of gas (e.g., in the conduit 140 or at the user interface 120). The oxygen sensor 250 can be, for example, an ultrasonic oxygen sensor, an electrical oxygen sensor, a chemical oxygen sensor, an optical oxygen sensor, a pulse oximeter (e.g., SpO₂ sensor), or any combination thereof.

The analyte sensor 252 can be used to detect the presence of an analyte in the exhaled breath of the user 20. The data output by the analyte sensor 252 can be stored in the memory device 204 and used by the control system 200 to determine the identity and concentration of any analytes in the breath of the user. In some implementations, the analyte sensor 252 is positioned near a mouth of the user to detect analytes in breath exhaled from the user's mouth. For example, when the user interface 120 is a facial mask that covers the nose and mouth of the user, the analyte sensor 252 can be positioned within the facial mask to monitor the user's mouth breathing. In other implementations, such as when the user interface 120 is a nasal mask or a nasal pillow mask, the analyte sensor 252 can be positioned near the nose of the user to detect analytes in breath exhaled through the user's nose. In still other implementations, the analyte sensor 252 can be positioned near the user's mouth when the user interface 120 is a nasal mask or a nasal pillow mask. In this implementation, the analyte sensor 252 can be used to detect whether any air is inadvertently leaking from the user's mouth and/or the user interface 120. In some implementations, the analyte sensor 252 is a volatile organic compound (VOC) sensor that can be used to detect carbon-based chemicals or compounds. In some implementations, the analyte sensor 252 can also be used to detect whether the user is breathing through their nose or mouth. For example, if the data output by an analyte sensor 252 positioned near the mouth of the user or within the facial mask (e.g., in implementations where the user interface 120 is a facial mask) detects the presence of an analyte, the control system 200 can use this data as an indication that the user is breathing through their mouth.

The moisture sensor 254 outputs data that can be stored in the memory device 204 and used by the control system 200. The moisture sensor 254 can be used to detect moisture in various areas surrounding the user (e.g., inside the conduit 140 or the user interface 120, near the user's face, near the connection between the conduit 140 and the user interface 120, near the connection between the conduit 140 and the respiratory therapy device 110, etc.). Thus, in some implementations, the moisture sensor 254 can be coupled to or integrated in the user interface 120 or in the conduit 140 to monitor the humidity of the pressurized air from the respiratory therapy device 110. In other implementations, the moisture sensor 254 is placed near any area where moisture levels need to be monitored. The moisture sensor 254 can also be used to monitor the humidity of the ambient environment surrounding the user, for example, the air inside the bedroom.

The LiDAR sensor 256 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 256 can measure and map an area extending 5 meters or more away from the sensor. The LiDAR data can be fused with point cloud data estimated by an electromagnetic RADAR sensor, for example. The LiDAR sensor(s) 256 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

In some implementations, the one or more sensors 210 also include a galvanic skin response (GSR) sensor, a blood flow sensor, a respiration sensor, a pulse sensor, a sphygmomanometer sensor, an oximetry sensor, a sonar sensor, a RADAR sensor, a blood glucose sensor, a color sensor, a pH sensor, an air quality sensor, a tilt sensor, a rain sensor, a soil moisture sensor, a water flow sensor, an alcohol sensor, or any combination thereof.

While shown separately in FIG. 1, any combination of the one or more sensors 210 can be integrated in and/or coupled to any one or more of the components of the system 10, including the respiratory therapy device 110, the user interface 120, the conduit 140, the humidifier 160, the control system 200, the user device 260, the activity tracker 270, or any combination thereof. For example, the microphone 220 and the speaker 222 can be integrated in and/or coupled to the user device 260 and the pressure sensor 212 and/or flow rate sensor 214 are integrated in and/or coupled to the respiratory therapy device 110. In some implementations, at least one of the one or more sensors 210 is not coupled to the respiratory therapy device 110, the control system 200, or the user device 260, and is positioned generally adjacent to the user 20 during the sleep session (e.g., positioned on or in contact with a portion of the user 20, worn by the user 20, coupled to or positioned on the nightstand, coupled to the mattress, coupled to the ceiling, etc.).

One or more of the respiratory therapy device 110, the user interface 120, the conduit 140, the display device 150, and the humidifier 160 can contain one or more sensors (e.g., a pressure sensor, a flow rate sensor, a microphone, or more generally any of the other sensors 210 described herein). These one or more sensors can be used, for example, to measure the air pressure and/or flow rate of pressurized air supplied by the respiratory therapy device 110.

The data from the one or more sensors 210 can be analyzed (e.g., by the control system 200) to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, a sleep state, an apnea-hypopnea index (AHI), or any combination thereof. The one or more events can include snoring, apneas, central apneas, obstructive apneas, mixed apneas, hypopneas, a mask leak, a cough, a restless leg, a sleeping disorder, choking, an increased heart rate, labored breathing, an asthma attack, an epileptic episode, a seizure, increased blood pressure, or any combination thereof. Many of these sleep-related parameters are physiological parameters, although some of the sleep-related parameters can be considered to be non-physiological parameters. Other types of physiological and non-physiological parameters can also be determined, either from the data from the one or more sensors 210, or from other types of data.

The user device 260 includes a display device 262. The user device 260 can be, for example, a mobile device such as a smartphone, a tablet computer, a gaming console, a smartwatch, a laptop computer, or the like. In some implementations, the user device 260 is a portable device, such as a smartphone, a tablet computer, a smartwatch, a laptop computer, etc. Alternatively, the user device 260 can be an external sensing system, a television (e.g., a smart television) or another smart home device (e.g., a smart speaker(s) such as Google Home™ Google Nest™, Amazon Echo™, Amazon Alexa™-enabled devices, etc.). In some implementations, the user device is a wearable device (e.g., a smartwatch). The display device 262 is generally used to display image(s) including still images, video images, or both. In some implementations, the display device 262 acts as a human-machine interface (HMI) that includes a graphic user interface (GUI) configured to display the image(s) and an input interface. The display device 262 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 260. In some implementations, one or more user devices can be used by and/or included in the system 10.

In some implementations, the system 10 also includes the activity tracker 270. The activity tracker 270 is generally used to aid in generating physiological data associated with the user. The activity tracker 270 can include one or more of the sensors 210 described herein, such as, for example, the motion sensor 218 (e.g., one or more accelerometers and/or gyroscopes), the PPG sensor 236, and/or the ECG sensor 238. The physiological data from the activity tracker 270 can be used to determine, for example, a number of steps, a distance traveled, a number of steps climbed, a duration of physical activity, a type of physical activity, an intensity of physical activity, time spent standing, a respiration rate, an average respiration rate, a resting respiration rate, a maximum he respiration art rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. In some implementations, the activity tracker 270 is coupled (e.g., electronically or physically) to the user device 260.

In some implementations, the activity tracker 270 is a wearable device that can be worn by the user, such as a smartwatch, a wristband, a ring, or a patch. For example, referring to FIG. 2, the activity tracker 270 is worn on a wrist of the user 20. The activity tracker 270 can also be coupled to or integrated a garment or clothing that is worn by the user. Alternatively still, the activity tracker 270 can also be coupled to or integrated in (e.g., within the same housing) the user device 260. More generally, the activity tracker 270 can be communicatively coupled with, or physically integrated in (e.g., within a housing), the control system 200, the memory device 204, the respiratory therapy system 100, and/or the user device 260.

In some implementations, the system 10 also includes the blood pressure device 280. The blood pressure device 280 is generally used to aid in generating cardiovascular data for determining one or more blood pressure measurements associated with the user 20. The blood pressure device 280 can include at least one of the one or more sensors 210 to measure, for example, a systolic blood pressure component and/or a diastolic blood pressure component.

In some implementations, the blood pressure device 280 is a sphygmomanometer including an inflatable cuff that can be worn by the user 20 and a pressure sensor (e.g., the pressure sensor 212 described herein). For example, in the example of FIG. 2, the blood pressure device 280 can be worn on an upper arm of the user 20. In such implementations where the blood pressure device 280 is a sphygmomanometer, the blood pressure device 280 also includes a pump (e.g., a manually operated bulb) for inflating the cuff. In some implementations, the blood pressure device 280 is coupled to the respiratory therapy device 110 of the respiratory therapy system 100, which in turn delivers pressurized air to inflate the cuff. More generally, the blood pressure device 280 can be communicatively coupled with, and/or physically integrated in (e.g., within a housing), the control system 200, the memory device 204, the respiratory therapy system 100, the user device 260, and/or the activity tracker 270.

In other implementations, the blood pressure device 280 is an ambulatory blood pressure monitor communicatively coupled to the respiratory therapy system 100. An ambulatory blood pressure monitor includes a portable recording device attached to a belt or strap worn by the user 20 and an inflatable cuff attached to the portable recording device and worn around an arm of the user 20. The ambulatory blood pressure monitor is configured to measure blood pressure between about every fifteen minutes to about thirty minutes over a 24-hour or a 48-hour period. The ambulatory blood pressure monitor may measure heart rate of the user 20 at the same time. These multiple readings are averaged over the 24-hour period. The ambulatory blood pressure monitor determines any changes in the measured blood pressure and heart rate of the user 20, as well as any distribution and/or trending patterns of the blood pressure and heart rate data during a sleeping period and an awakened period of the user 20. The measured data and statistics may then be communicated to the respiratory therapy system 100.

The blood pressure device 280 maybe positioned external to the respiratory therapy system 100, coupled directly or indirectly to the user interface 120, coupled directly or indirectly to a headgear associated with the user interface 120, or inflatably coupled to or about a portion of the user 20. The blood pressure device 280 is generally used to aid in generating physiological data for determining one or more blood pressure measurements associated with a user, for example, a systolic blood pressure component and/or a diastolic blood pressure component. In some implementations, the blood pressure device 280 is a sphygmomanometer including an inflatable cuff that can be worn by a user and a pressure sensor (e.g., the pressure sensor 212 described herein).

In some implementations, the blood pressure device 280 is an invasive device which can continuously monitor arterial blood pressure of the user 20 and take an arterial blood sample on demand for analyzing gas of the arterial blood. In some other implementations, the blood pressure device 280 is a continuous blood pressure monitor, using a radio frequency sensor and capable of measuring blood pressure of the user 20 once very few seconds (e.g., every 3 seconds, every 5 seconds, every 7 seconds, etc.) The radio frequency sensor may use continuous wave, frequency-modulated continuous wave (FMCW with ramp chirp, triangle, sinewave, etc.), other schemes such as PSK, FSK etc., pulsed continuous wave, and/or spread in ultra-wideband ranges (which may include spreading, PRN codes or impulse systems).

While the control system 200 and the memory device 204 are described and shown in FIG. 1 as being a separate and distinct component of the system 10, in some implementations, the control system 200 and/or the memory device 204 are integrated in the user device 260 and/or the respiratory therapy device 110. Thus, the control system 200 and/or the memory device 204 can be disposed within the housing 112 of the respiratory therapy device 110. Alternatively, in some implementations, the control system 200 or a portion thereof (e.g., the processor 202) can be located in a cloud (e.g., integrated in a server, integrated in an Internet of Things (IOT) device, connected to the cloud, be subject to edge cloud processing, etc.), located in one or more servers (e.g., remote servers, local servers, etc., or any combination thereof.

While system 10 is shown as including all the components described above, more or fewer components can be included in a system according to implementations of the present disclosure. For example, a first alternative system includes the control system 200, the memory device 204, and at least one of the one or more sensors 210 and does not include the respiratory therapy system 100. As another example, a second alternative system includes the control system 200, the memory device 204, at least one of the one or more sensors 210, and the user device 260. As yet another example, a third alternative system includes the control system 200, the memory device 204, the respiratory therapy system 100, at least one of the one or more sensors 210, and the user device 260. Thus, various systems can be formed using any portion or portions of the components shown and described herein and/or in combination with one or more other components.

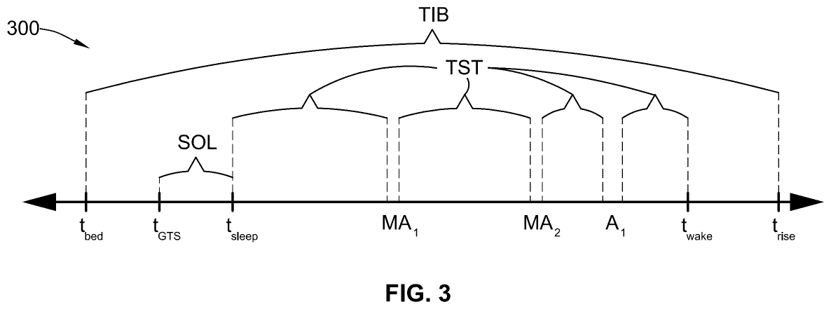

Referring now to FIG. 3, as used herein, a sleep session can be defined multiple ways. For example, a sleep session can be defined by an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, the user can manually define the beginning of a sleep session and/or manually terminate a sleep session. For example, the user can select (e.g., by clicking or tapping) one or more user-selectable element that is displayed on the display device 262 of the user device 260 (FIG. 1) to manually initiate or terminate the sleep session.

Generally, the sleep session includes any point in time after the user has laid or sat down in the bed (or another area or object on which they intend to sleep), and has turned on the respiratory therapy device 110 and donned the user interface 120. The sleep session can thus include time periods (i) when the user is using the respiratory therapy system 100, but before the user attempts to fall asleep (for example when the user lays in the bed reading a book); (ii) when the user begins trying to fall asleep but is still awake; (iii) when the user is in a light sleep (also referred to as stage 1 and stage 2 of non-rapid eye movement (NREM) sleep); (iv) when the user is in a deep sleep (also referred to as slow-wave sleep, SWS, or stage 3 of NREM sleep); (v) when the user is in rapid eye movement (REM) sleep; (vi) when the user is periodically awake between light sleep, deep sleep, or REM sleep; or (vii) when the user wakes up and does not fall back asleep. The sleep session may also be referred to as a therapy session, or may comprise a therapy session, which can be understood to be the period of time within the sleep session during which the individual is engaged in respiratory therapy (e.g., the use of a respiratory therapy system).

The sleep session is generally defined as ending once the user removes the user interface 120, turns off the respiratory therapy device 110, and gets out of bed. In some implementations, the sleep session can include additional periods of time, or can be limited to only some of the above-disclosed time periods. For example, the sleep session can be defined to encompass a period of time beginning when the respiratory therapy device 110 begins supplying the pressurized air to the airway or the user, ending when the respiratory therapy device 110 stops supplying the pressurized air to the airway of the user, and including some or all of the time points in between, when the user is asleep or awake.

FIG. 3 illustrates an exemplary timeline 300 for a sleep session. The timeline 300 includes an enter bed time (t_{bed}), a go-to-sleep time (t_{GTS}), an initial sleep time (tₛₗₑₑₚ), a first micro-awakening MA₁, a second micro-awakening MA₂, an awakening A, a wake-up time (t_{wake}), and a rising time (tᵣᵢₛₑ).

The enter bed time t_{bed} is associated with the time that the user initially enters the bed (e.g., bed 40 in FIG. 2) prior to falling asleep (e.g., when the user lies down or sits in the bed). The enter bed time t_{bed} can be identified based at least in part on a bed threshold duration to distinguish between times when the user enters the bed for sleep and when the user enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the user initially enters any location for sleeping (e.g., a couch, a chair, a sleeping bag, etc.).

The go-to-sleep time (GTS) is associated with the time that the user initially attempts to fall asleep after entering the bed (t_{bed}). For example, after entering the bed, the user may engage in one or more activities to wind down prior to trying to sleep (e.g., reading, watching TV, listening to music, using the user device 260, etc.). The initial sleep time (tₛₗₑₑₚ) is the time that the user initially falls asleep. For example, the initial sleep time (tₛₗₑₑₚ) can be the time that the user initially enters the first non-REM sleep stage.

The wake-up time t_{wake} is the time associated with the time when the user wakes up without going back to sleep (e.g., as opposed to the user waking up in the middle of the night and going back to sleep). The user may experience one of more unconscious microawakenings (e.g., microawakenings MA₁ and MA₂) having a short duration (e.g., 5 seconds, 10 seconds, 30 seconds, 1 minute, etc.) after initially falling asleep. In contrast to the wake-up time t_{wake}, the user goes back to sleep after each of the microawakenings MA₁ and MA₂. Similarly, the user may have one or more conscious awakenings (e.g., awakening A) after initially falling asleep (e.g., getting up to go to the bathroom, attending to children or pets, sleep walking, etc.). However, the user goes back to sleep after the awakening A. Thus, the wake-up time t_{wake} can be defined, for example, based at least in part on a wake threshold duration (e.g., the user is awake for at least 15 minutes, at least 20 minutes, at least 30 minutes, at least 1 hour, etc.).

Similarly, the rising time tᵣᵢₛₑ is associated with the time when the user exits the bed and stays out of the bed with the intent to end the sleep session (e.g., as opposed to the user getting up during the night to go to the bathroom, to attend to children or pets, sleep walking, etc.). In other words, the rising time tᵣᵢₛₑ is the time when the user last leaves the bed without returning to the bed until a next sleep session (e.g., the following evening). Thus, the rising time tᵣᵢₛₑ can be defined, for example, based at least in part on a rise threshold duration (e.g., the user has left the bed for at least 15 minutes, at least 20 minutes, at least 30 minutes, at least 1 hour, etc.). The enter bed time t_{bed} time for a second, subsequent sleep session can also be defined based at least in part on a rise threshold duration (e.g., the user has left the bed for at least 4 hours, at least 6 hours, at least 8 hours, at least 12 hours, etc.).

As described above, the user may wake up and get out of bed one more times during the night between the initial t_{bed} and the final tᵣᵢₛₑ. In some implementations, the final wake-up time t_{wake} and/or the final rising time tᵣᵢₛₑ that are identified or determined based at least in part on a predetermined threshold duration of time subsequent to an event (e.g., falling asleep or leaving the bed). Such a threshold duration can be customized for the user. For a standard user which goes to bed in the evening, then wakes up and goes out of bed in the morning any period (between the user waking up (t_{wake}) or raising up (tᵣᵢₛₑ), and the user either going to bed (t_{bed}), going to sleep (t_{GTS}) or falling asleep (tₛₗₑₑₚ) of between about 12 and about 18 hours can be used. For users that spend longer periods of time in bed, shorter threshold periods may be used (e.g., between about 8 hours and about 14 hours). The threshold period may be initially selected and/or later adjusted based at least in part on the system monitoring the user's sleep behavior.

The total time in bed (TIB) is the duration of time between the time enter bed time t_{bed} and the rising time tᵣᵢₛₑ. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, as shown in the timeline 300, the total sleep time (TST) spans between the initial sleep time tₛₗₑₑₚ and the wake-up time t_{wake}, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 5 minutes, etc. The persistent total sleep time is a measure of sustained sleep, and smooths the sleep-wake hypnogram. For example, when the user is initially falling asleep, the user may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (t_{bed}) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (t_{GTS}) and ending at the wake-up time (t_{wake}). In some implementations, a sleep session is defined as starting at the go-to-sleep time (t_{GTS}) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

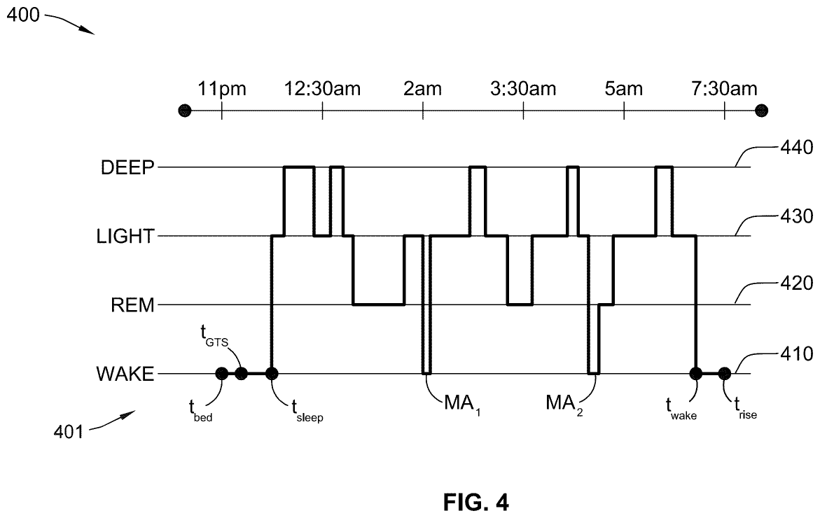

Referring to FIG. 4, an exemplary hypnogram 400 corresponding to the timeline 300 of FIG. 3, according to some implementations, is illustrated. As shown, the hypnogram 400 includes a sleep-wake signal 401, a wakefulness stage axis 410, a REM stage axis 420, a light sleep stage axis 430, and a deep sleep stage axis 440. The intersection between the sleep-wake signal 401 and one of the axes 410-440 is indicative of the sleep stage at any given time during the sleep session.

The sleep-wake signal 401 can be generated based at least in part on physiological data associated with the user (e.g., generated by one or more of the sensors 210 described herein). The sleep-wake signal can be indicative of one or more sleep stages, including wakefulness, relaxed wakefulness, microawakenings, a REM stage, a first non-REM stage, a second non-REM stage, a third non-REM stage, or any combination thereof. In some implementations, one or more of the first non-REM stage, the second non-REM stage, and the third non-REM stage can be grouped together and categorized as a light sleep stage or a deep sleep stage. For example, the light sleep stage can include the first non-REM stage and the deep sleep stage can include the second non-REM stage and the third non-REM stage. While the hypnogram 400 is shown in FIG. 4 as including the light sleep stage axis 430 and the deep sleep stage axis 440, in some implementations, the hypnogram 400 can include an axis for each of the first non-REM stage, the second non-REM stage, and the third non-REM stage. In other implementations, the sleep-wake signal can also be indicative of a respiration signal, a respiration rate, an inspiration amplitude, an expiration amplitude, an inspiration-expiration amplitude ratio, an inspiration-expiration duration ratio, a number of events per hour, a pattern of events, or any combination thereof. Information describing the sleep-wake signal can be stored in the memory device 204.

The hypnogram 400 can be used to determine one or more sleep-related parameters, such as, for example, a sleep onset latency (SOL), wake-after-sleep onset (WASO), a sleep efficiency (SE), a sleep fragmentation index, sleep blocks, or any combination thereof.

The sleep onset latency (SOL) is defined as the time between the go-to-sleep time (t_{GTS}) and the initial sleep time (tₛₗₑₑₚ). In other words, the sleep onset latency is indicative of the time that it took the user to actually fall asleep after initially attempting to fall asleep. In some implementations, the sleep onset latency is defined as a persistent sleep onset latency (PSOL). The persistent sleep onset latency differs from the sleep onset latency in that the persistent sleep onset latency is defined as the duration time between the go-to-sleep time and a predetermined amount of sustained sleep. In some implementations, the predetermined amount of sustained sleep can include, for example, at least 10 minutes of sleep within the second non-REM stage, the third non-REM stage, and/or the REM stage with no more than 2 minutes of wakefulness, the first non-REM stage, and/or movement therebetween. In other words, the persistent sleep onset latency requires up to, for example, 8 minutes of sustained sleep within the second non-REM stage, the third non-REM stage, and/or the REM stage. In other implementations, the predetermined amount of sustained sleep can include at least 10 minutes of sleep within the first non-REM stage, the second non-REM stage, the third non-REM stage, and/or the REM stage subsequent to the initial sleep time. In such implementations, the predetermined amount of sustained sleep can exclude any micro-awakenings (e.g., a ten second micro-awakening does not restart the 10-minute period).

The wake-after-sleep onset (WASO) is associated with the total duration of time that the user is awake between the initial sleep time and the wake-up time. Thus, the wake-after-sleep onset includes short and micro-awakenings during the sleep session (e.g., the micro-awakenings MA₁ and MA₂ shown in FIG. 4), whether conscious or unconscious. In some implementations, the wake-after-sleep onset (WASO) is defined as a persistent wake-after-sleep onset (PWASO) that only includes the total durations of awakenings having a predetermined length (e.g., greater than 10 seconds, greater than 30 seconds, greater than 60 seconds, greater than about 5 minutes, greater than about 10 minutes, etc.) The sleep efficiency (SE) is determined as a ratio of the total time in bed (TIB) and the total sleep time (TST). For example, if the total time in bed is 8 hours and the total sleep time is 7.5 hours, the sleep efficiency for that sleep session is 93.75%. The sleep efficiency is indicative of the sleep hygiene of the user. For example, if the user enters the bed and spends time engaged in other activities (e.g., watching TV) before sleep, the sleep efficiency will be reduced (e.g., the user is penalized). In some implementations, the sleep efficiency (SE) can be calculated based at least in part on the total time in bed (TIB) and the total time that the user is attempting to sleep. In such implementations, the total time that the user is attempting to sleep is defined as the duration between the go-to-sleep (GTS) time and the rising time described herein. For example, if the total sleep time is 8 hours (e.g., between 11 PM and 7 AM), the go-to-sleep time is 10:45 PM, and the rising time is 7:15 AM, in such implementations, the sleep efficiency parameter is calculated as about 94%.

The fragmentation index is determined based at least in part on the number of awakenings during the sleep session. For example, if the user had two micro-awakenings (e.g., micro-awakening MA₁ and micro-awakening MA₂ shown in FIG. 4), the fragmentation index can be expressed as 2. In some implementations, the fragmentation index is scaled between a predetermined range of integers (e.g., between 0 and 10).

The sleep blocks are associated with a transition between any stage of sleep (e.g., the first non-REM stage, the second non-REM stage, the third non-REM stage, and/or the REM) and the wakefulness stage. The sleep blocks can be calculated at a resolution of, for example, 30 seconds.

In some implementations, the systems and methods described herein can include generating or analyzing a hypnogram including a sleep-wake signal to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof based at least in part on the sleep-wake signal of a hypnogram.

In other implementations, one or more of the sensors 210 can be used to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof, which in turn define the sleep session. For example, the enter bed time t_{bed} can be determined based at least in part on, for example, data generated by the motion sensor 218, the microphone 220, the camera 232, or any combination thereof. The go-to-sleep time can be determined based at least in part on, for example, data from the motion sensor 218 (e.g., data indicative of no movement by the user), data from the camera 232 (e.g., data indicative of no movement by the user and/or that the user has turned off the lights), data from the microphone 220 (e.g., data indicative of the using turning off a TV), data from the user device 260 (e.g., data indicative of the user no longer using the user device 260), data from the pressure sensor 212 and/or the flow rate sensor 214 (e.g., data indicative of the user turning on the respiratory therapy device 110, data indicative of the user donning the user interface 120, etc.), or any combination thereof.

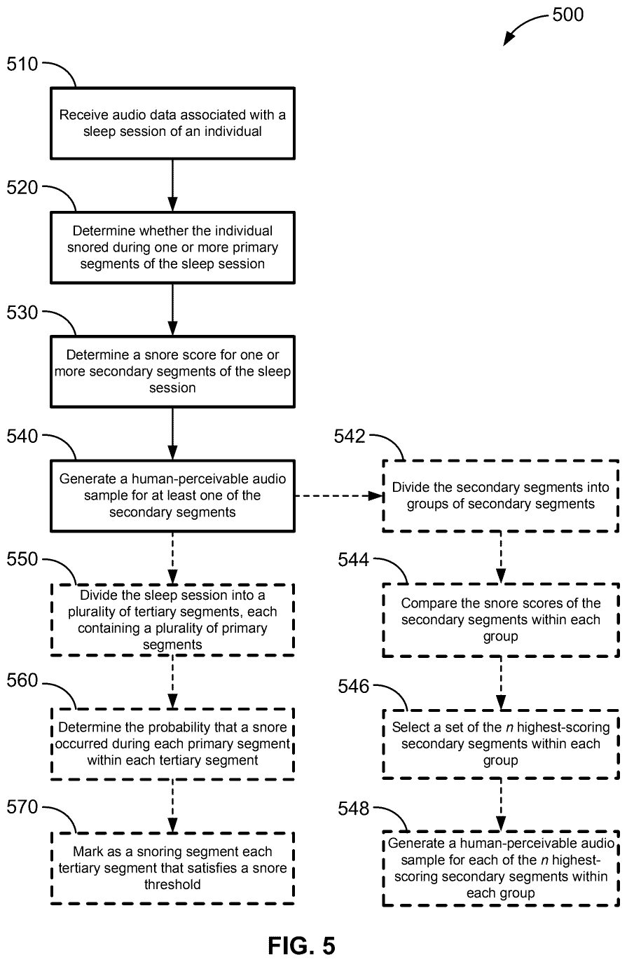

FIG. 5 shows a flowchart of a method 500 for analyzing sounds made by an individual during a sleep session. At step 510 of method 500, audio data associated with the sleep session can be generated and/or received. Generally, a variety of different sounds and/or noises can occur in and around the room where the individual is located during the sleep session (e.g., the individual's bedroom). These sounds will generally include any snoring sounds made by the individual, but may also include other types of sounds as well, such as other sounds made by the individual (e.g., coughing noises, choking noises, sleep-talking, etc.), sounds made by the individual's bed partner, sounds caused by movement of the individual (and/or the individual's bed partner), sounds occurring outside of the individual's dwelling (e.g., sounds made by cars driving on the road, sounds made animals, etc.), and other sounds.

Audio data that is representative of any of these noises can be generated and/or received. For example, method 500 may be implemented by a system (such as system 10) that includes one or more audio sensors (such as microphone 220 of system 10). The audio sensors of the system can detect these sounds and generate audio data that is representative of these sounds. In other examples, the system that implements method 500 includes an electronic interface that is configured to receive data from external sources. In these examples, the audio data can be generated separately, and then received by the electronic interface of the system. In any of these examples, the audio data can be stored in a memory device of the system (such as memory device 204 of system 10).

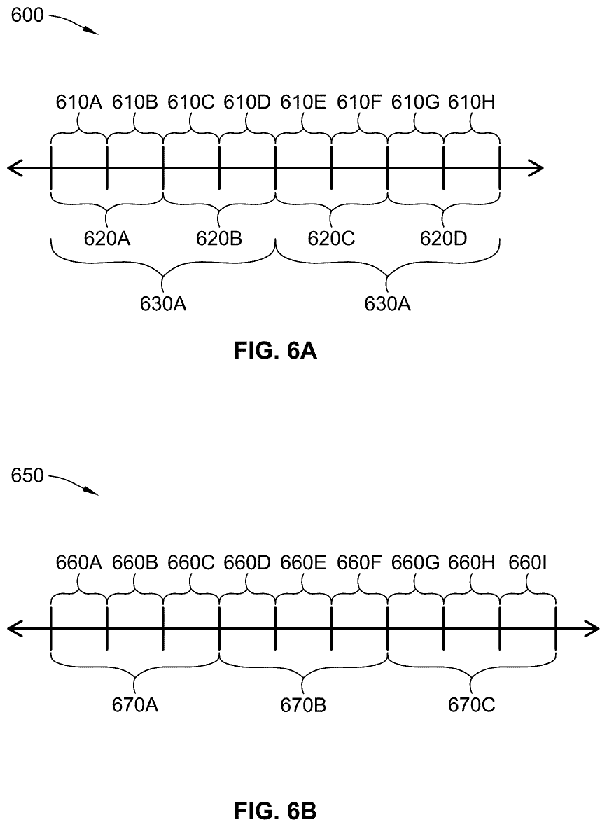

Step 520 of method 500 includes determining whether one or more snoring sounds were made by the individual during each of at one or more primary segments of the sleep session. Generally, the sleep session can be divided into a plurality of small durations of time, referred to as primary segments, which can be individually analyzed. FIG. 6A shows a timeline of an exemplary sleep session 600. As shown, the sleep session 600 is divided into a plurality of primary segments 610A-610H. While only these eight primary segments are shown, a sleep session can have any number of primary segments. Generally, each of the primary segments 610A-610H of the sleep session 600 will have the same fixed duration, which may be any number of seconds, minutes, hours, etc. However, in some implementations, the duration of the primary segments may vary. In some implementations, each of the primary segments 610A-610H has a duration of about one second. Thus, if a sleep session in one of these implementations has a total duration of 8 hours, the sleep session will be divided into 28,800 primary segments. In most implementations, the sleep session is divided into at least two primary segments, and thus step 520 includes determining whether one or more snoring sound were made by the individual during each of at least two or more primary segments of the sleep session. However, in some implementations, the sleep session may comprise one primary segment.

Referring back to FIG. 5, the audio data for each of these primary segments can be analyzed to determine whether any snoring sounds were made by the individual during each of these primary segments. Generally, step 520 includes analyzing each of the primary segments of the sleep session individually. Thus, step 520 can include determining, for each respective primary segment, whether any snoring sounds were made by the individual during that respective primary segment (e.g., detecting snoring sounds that were made by the individual during that respective primary segment). In some implementations though, not all of the primary segments are analyzed. For example, primary segments near the beginning and/or the end of the sleep session may be discarded, as it may be likely that the individual was awake during these primary segments. In general, step 520 includes determining whether any snoring sounds were made by the individual during each of at least two of the primary segments of the sleep session.

In some implementations, determining whether one or more snoring sounds were made by the individual at step 520 includes determining the probability that one or more snoring sounds occurred during a given primary segment of the sleep session (e.g., the probability that one or more of the noises detected in the primary segment was a snoring sound). The probability can be represented as a decimal number between 0 and 1, (e.g., 0.2, 0.5, 0.8, etc.), as a whole number between 0 and 100 (e.g., 20%, 50%, 80%, etc.), or any other suitable manner. In some implementations, the audio data associated with a respective primary segment can be input into a trained algorithm (such as a machine learning model) that has been trained to output the probability that one or more snoring sounds occurred during the respective primary segment. The term “snore probability” is used herein to refer to the probability that one or more snoring sounds occurred during a given timeframe comprising one or more primary segments. Thus, the snore probability of a primary segment refers to the probability that one or more snoring sounds occurred during that primary segment.

In some implementations, analyzing the acoustic data to determine whether one or more snoring sounds occurred during a primary segment of the sleep session can include identifying an acoustic signature in the audio data for the primary segment that is indicative of a snoring sound. The acoustic signature can be any feature or combination of features that is unique to a snoring sound made by the individual. An acoustic pattern could be any type of periodic (e.g., repeating) feature or features in the acoustic data that results from a certain type of noise. In some implementations, the acoustic pattern resulting from a certain type of noise is the acoustic signature of that noise. In other implementations, the acoustic pattern resulting from a certain type of noise may be shared between multiple types of noise. The acoustic data can be analyzed to identify this acoustic pattern or acoustic signature of any snoring sounds. In some implementations, the acoustic pattern or signature can be indicative of the probability that a given primary segment of the sleep session contains one or more snoring sounds.

In some implementations, analyzing the acoustic data includes generating time-domain measurements, such as a measurement representing the power of the detected noise versus time. Generally, the power of a detected noise (e.g., the power of an acoustic signal generated from the acoustic data that represents the noise) is representative of and/or correlates with how loud the noise is (which could also be measured as a specific intensity/volume and represented with a dB value in some implementations). The power of the detected noise is generally measured in arbitrary units. The power of the noise can be analyzed to detect an acoustic pattern or acoustic signature indicative of snoring sounds. For example, a snoring sound may be represented by an acoustic signal with a periodic pattern in the time-domain measurement, e.g., a plot of the power of the detected noise versus time where the power repeatedly increases and decreases over time. The power may vary (e.g., increase and decrease, wax and wane, etc.) generally over a variety of different time periods, and still indicate a snoring sound. In some cases, the power increases and decreases several times (e.g., two to five times) per second. This periodic power pattern can be identified to aid in determining whether a snoring sound occurred during the primary segment. Additionally or alternatively, the value of the power of the snoring sound can be determined from the acoustic signal. The value of the power may itself be used to determine whether a snoring sound occurred. The baseline power can be determined by analyzing audio data when it is known that no snoring sounds and generally little to no other noises were occurring (e.g., during the beginning of the sleep session prior to the individual falling asleep, during a calibration period within the sleep session, etc.). In some implementations, the time-domain measurements can be indicative of the probability that a given primary segment of the sleep session contains one or more snoring sounds.

In some implementations, analyzing the acoustic data includes generating frequency-domain measurements, such as a frequency spectrum that represents the power of the detected noise versus frequency. In some implementations, a spectrogram can be obtained by taking the Fourier transform of the measurement representing the power of the detected noise versus time. The spectrogram can be analyzed to identify various features representing a snoring sound, which may have a distinct frequency, or be composed of multiple frequencies within distinct frequency range. Thus, in some implementations, the frequency-domain measurements can be indicative of the probability that a given primary segment of the sleep session contains one or more snoring sounds.

In some implementations, analyzing the acoustic data includes utilizing cepstral analysis. A cepstrum can be considered as a spectrum of a spectrum, and can be obtained in some implementations by taking the inverse Fourier Transform of the logarithm of the frequency spectrum. In some implementations, the frequency spectrum is plotted on the mel scale. The mel scale is a warped version of a linear frequency scale, where the difference between consecutive frequency intervals is not equally-spaced as the frequency increases. The mel scale generally approximates the response of the human auditory scale more accurately than a linear frequency scale. After the mel-frequency cepstrum has been generated, the mel-frequency cepstral coefficients can be determined from the mel-frequency cepstrum by utilizing a discrete cosine transform of the mel-frequency spectrum to obtain mel-frequency cepstrum coefficients. The mel-frequency cepstral coefficients are a set of amplitudes of the components of the spectrum.

In some implementations, a set of mel-frequency cepstral coefficients are generated for each respective primary segment of the sleep session that is being analyzed. The mel-frequency cepstral coefficients for each respective primary segment can be input into a trained algorithm (e.g., a machine learning model such as a convolutional neural network), which is configured to determine whether any snoring sounds occurred during the respective primary segment. In some implementations, the trained model analyzes the mel-frequency cepstral coefficients to determine the probability that one or more snoring sounds occurred during the respective primary segment.

In some implementations, analyzing the audio data can include filtering out portions of the audio data that are not likely to represent snoring sounds. For example, most snoring sounds will have a frequency that is generally within a known frequency range. Audio data that represent sounds having a frequency greater than a predetermined threshold frequency can be filtered out, so that the amount of audio data that needs to be analyzed to detect snoring sounds is reduced. In some implementations, the threshold frequency is generally equal to the higher end of the known frequency range. In other implementations, the threshold frequency is equal to the higher end of the known frequency range plus some buffer amount, to reduce the chance that audio data representing a snoring sound is inadvertently filtered out. For example, snoring sounds generally have a frequency between about 200 Hz and about 2 kHz. The threshold frequency could be set to 2 kHz so that the audio data representing sounds having a frequency higher than about 2 kHz can be removed. The threshold frequency could also be set to, for example, 5 kHz, so that audio data is only removed if it represents sounds having a frequency higher than about 5 kHz, which reduces the chance that audio data representing a snoring sound is inadvertently removed.

In some implementations, both an upper threshold frequency and a lower threshold frequency can be utilized. Audio data representing sounds having a frequency higher than the upper threshold frequency or lower than the lower threshold frequency can be removed. The upper threshold frequency could be 2 kHz, 5 kHz, or any other suitable frequency. The lower threshold frequency could be 200 Hz, 100 Hz, or any other suitable frequency.

A variety of different trained algorithms can be used to determine whether one or more snoring sounds were made by the individual during a primary segment of the sleep session (e.g., to determine the probability that one or more snoring sounds occurred during a primary segment). As noted above, the determination of whether a snoring sound was made can be made based at least in part on mel-frequency cepstral coefficients that are input into the trained algorithm.

In some implementations, the algorithm is a machine learning model that has been trained using labeled audio data. The audio data can be collected from a variety of different sources, and may include audio data collected from polysomnography (e.g., a sleep study), audio data collected from other individuals using a respiratory therapy system, etc. The audio data can then be divided into segments, and audio samples for each segment can be generated. The audio samples can be analyzed by humans (e.g., listened to) and then labeled. The label of a respective audio segment can indicate what types of sounds occurred during that segment of audio data.

In some implementations, each segment of audio data is labeled as either including a snoring sound or not including a snoring sound. In other implementations, more detailed labels can be used, which may indicate different types of snoring sounds and/or other noises. For example, each segment of audio data could be labeled as including (i) no snoring or breathing sounds, (ii) breathing sounds, (iii) heavy breathing sounds, (iv) snoring sounds, and/or (v) heavy snoring sounds. Other labeling schemes could also be used. Once the segments of audio data are labeled, they can be used to train the machine learning model.

Using humans to listen to the segments of audio data and label the segments as containing a snoring sound or not (and/or containing variations of snoring sounds, variations of other noises, etc.) results in the model being more representative of how snoring sounds are actually perceived by humans. Humans can label the segments of audio data based on whether they perceive there to be a snoring sound (and/or a heavy snoring sound, a breathing sound, a heavy breathing sound, etc.). By training the machine learning model with segments of audio data that has been labeled based on human perception of the noises within the segments, the snore probabilities that are output by the trained model will more accurately reflect how snoring sounds made by the individual will be perceived by a human (such as the individual's bed partner). In other implementations, machine-labeled audio data, some or all of which may have been confirmed by a human, can be used to train the machine learning model.

In some implementations, the machine learning model can additionally be trained using audio data that is known to contain noises other than snoring and/or breathing, such as music, speech, common external noises such as vehicle noises or animal noises, etc. Training the model using this non-snoring and non-breathing data and improve the model's ability to differentiate between snoring and breathing noises, and non-snoring and non-breathing noises.

Step 530 of method 500 includes determining a snore score for each of one or more secondary segments of the sleep session. In addition to the primary segments, the sleep session can be further divided into one or more secondary segments, where each secondary segments includes a plurality of primary segments. In the example shown in FIG. 6A, the sleep session 600 is divided into a plurality of secondary segments, 620A, 620B, 620C, and 620D. Each of these secondary segments is illustrated as containing two of the primary segments but, as noted below, may contain more than two of the primary segments. Secondary segment 620A includes primary segment 610A and primary segment 610B. Secondary segment 620B includes primary segment 610C and primary segment 610D. Secondary segment 620C includes primary segment 610E and primary segment 610F. Secondary segment 620D includes primary segment 610G and primary segment 610H.

While the sleep session 600 in FIG. 6A is illustrated with eight primary segments 610A-610H and four secondary segments 620A-620D, a sleep session can have any number of primary segments, any number of secondary segments, and any number of primary segments within each secondary segment. In some implementations, each secondary segment has an equal number of primary segments. In some implementations, some secondary segments may have differing numbers of primary segments. In some implementations, each of the primary segments and each of the secondary segments has a fixed duration. Generally, the total duration of a given secondary segment is equal to the sum of the duration of each of the primary segments contained within the secondary segment. In the example sleep session 600 illustrated in FIG. 6A, each of the secondary segments 620A-620D has a duration that is twice as long as the duration of each of the primary segments 610A-610H. Thus, if each of the primary segments 610A-610H has a duration of one second, each of the secondary segments will have a duration of two seconds. However, a given sleep session can be divided into primary and secondary segments in different ways. For example, in some implementations, each of the secondary segments of a sleep session has a duration of about 30 seconds. If each of the primary segments of this sleep session has a duration of about 1 second, then each secondary segment will contain 30 primary segments therein. If each of the primary segments of this sleep session has a duration of about 2 seconds, then each secondary segment will contain 15 primary segments therein.

Referring back to FIG. 5, determining the snore score for each respective secondary segment at step 530 generally includes analyzing each of the primary segments contained with the respective secondary segment, and is based at least in part on the determination of whether one or more snoring sounds occurred within the primary segments. In implementations where determining whether a snoring sound occurred within a primary segment includes determining the probability that a snoring sound occurred during the primary segment, the snore score for each respective secondary segment is based at least in part on the probability that at least one snoring sound occurred during the secondary segment (e.g., the snore probability of the secondary segment).

In some implementations, the snore score for each respective secondary segment is based at least in part on an average (also referred to as an arithmetic mean) of the individual snore probabilities across all of the primary segments contained within the respective secondary segment. For example, if a given secondary segment contains fifteen primary segments, the average snore probability for that secondary segment is the sum of the fifteen individual snore probabilities divided by fifteen. In other implementations, the root mean square (RMS) value of the individual snore probabilities of the primary segments contained within the respective secondary segment is used, instead of the average. The RMS value is also referred to as the quadratic mean.

In some implementations, the snore score for each respective secondary segment is additionally or alternatively based on the power of an acoustic signal representing any sounds detected in the respective secondary segment (such detected sounds can include one or more snoring sounds occurring during the respective secondary segment). In some of these implementations, the snore score is based at least in part on an RMS value of the power of sounds detected in the primary segments contained within the respective secondary segment. In a first example of these implementations, the RMS power value is separately determined for each primary segment within the respective secondary segment, and the individual RMS power values are then averaged together to generate a power value for the secondary segment. The audio data corresponding to the primary segment will generally be representative of a plurality of individual power values across the primary segment, which can be used to determine the RMS power value for that primary segment.

In a second example of these implementations, a single RMS power value is determined across all of the primary segments within the respective secondary segments, Thus, if a respective secondary segment contains fifteen primary segments, the RMS value of the power for the respective secondary segment is the RMS value of the power across of the fifteen individual snore probabilities for the fifteen primary segments. Generally, the RMS power value (whether for a primary segment or a secondary segment) can be determined by squaring the individual power values (e.g., the individual power values across a primary segment and/or a secondary segment), determining the average (e.g., the arithmetic mean) of the squared power values, and then determining the square root of that average.

In further implementations, the snore score for each respective secondary segment is based at least in part on the average power value for the secondary segment, e.g., the average of the individual power values within the respective secondary segment.

In some implementations, the snore score for a respective secondary segment is based on both the snore probability for the respective secondary segment, and the power of sounds detected in the respective secondary segment. Generally in these implementations, the snore score for the respective secondary segment is based on (i) the average snore probability across the primary segments contained within the respective secondary segment, and (ii) the RMS value of the power of sounds detected in each of the primary segments contained within the respective secondary segment. In some of these implementations, the snore score for the respective secondary segment is a weighted sum of the average snore probability and the RMS power value. In some implementations, the weighted sum for each respective secondary segment can be determined according to (0.5*Power_{RMS}+\tilde{P}ₛₙₒᵣₑ)/1.5. Here, Power_{RMS} is the RMS value of the power of any sounds detected in each of the primary segments contained within the respective secondary segment, and \tilde{P}ₛₙₒᵣₑ is the average snore probability across the primary segments contained within the respective secondary segment. As can be seen, the average snore probability across the primary segments is weighted about twice as much as the RMS power of the primary segments. Generally, both Power_{RMS} and \tilde{P}ₛₙₒᵣₑ will have a value that is greater than or equal to 0, and less than or equal to 1. Thus, \tilde{P}ₛₙₒᵣₑ+0.5*Power_{RMS} has a maximum value of 1.5, and dividing that value by 1.5 will normalize the snore score of the secondary segment to a value between 0 and 1.

Step 540 of method 500 includes generating a human-perceivable audio sample of at least one of the secondary segments of the sleep session. Many individuals tend to be resistant to the idea that they snore while sleeping, and are thus in turn resistant to the idea that they may be suffering from a condition that would benefit from treatment (e.g., SDB or OSA). The playback of the audio samples is intended to provide the individual with a clear indication of their snoring. The presently disclosed systems and methods allow the individual (or a third party, such as a healthcare provider) to listen to the audio sample in order to hear what their snoring sounds like. The generated audio samples can be played back on any suitable device. For example, the audio samples can be played on a user device of the individual (such as the user device 260 of the system 10), which could be a smartphone, a tablet computer, a smart watch, etc. The audio samples can also, or instead, be sent to a third party (such as a healthcare provider).

In some implementations, step 540 includes sub-steps 542-548, where specific secondary segments of the sleep session are selected based on their snore score. The snore score is generally based on both (i) the probability that one or more snoring sounds occurred and (ii) the power of any sounds detected. As such, secondary segments with higher snore scores are secondary segments where it is likely that the individual was audibly snoring. Audio samples of these secondary segments are thus the most well-suited to convince the individual that they do snore while they are sleeping, and generally provide the individual with the highest-quality audio data of their snoring during the sleep session.

At sub-step 542, the secondary segments of the sleep session are sorted into a plurality of groups of secondary segments. For example, referring to the sleep session 600 illustrated in FIG. 6A, the four secondary segments 620A, 620B, 620C, and 620D are sorted into two groups 630A and 630B. Group 630A includes secondary segments 620A and 620B, and group 630B includes secondary segments 620C and 620D. While FIG. 6A shows only two groups of two secondary segments each, a sleep session can be divided into any number of groups of secondary segments, and each group of secondary segments can have any number of secondary segments contained therein.

In some implementations, each group of secondary segments will contain enough secondary segments so that the sum of the duration of all of the secondary segments within a given group (e.g., the duration of the group of secondary segments) is equal to at least one minute. In some implementations, each group of secondary segments contains enough secondary segments so that the sum of the duration of the secondary segments contained within each group is equal to about one hour. In some of these implementations where each of the secondary segments has a duration of about 30 seconds, each group of secondary segments would thus contain about 120 secondary segments. In some implementations, each group contains the same number of secondary segments. In other implementations, some groups may include different numbers of secondary segments.

At sub-step 544, the snore score of each secondary segment within each group of secondary segments is compared. At sub-step 546, a set of the n highest-scoring secondary segments within each group are selected. At sub-step 548, a human-perceivable audio sample of each of the selected secondary segments (e.g., each of the n highest-scoring secondary segments within each group) is generated. Thus, certain secondary segments within each group having a sufficiently high score (indicating that the individual was audibly snoring in these segments) can be identified, and audio samples of these secondary segments can be generated.

Generally, n is an integer value that is greater than or equal to one, but is often equal to at least two, such that multiple audio samples will be generated from each group. In some implementations, n is equal to two. In some implementations, n is equal to five. In some implementations, n is equal to ten. In some implementations, the number of secondary segments selected from each group may vary across groups. For example, selecting the secondary segments may include selecting secondary segments having a snore score that satisfies a predetermined threshold snore score. If more than n secondary segments within a given group satisfy the threshold snore score (e.g., have a snore score that is greater than or equal to the threshold snore score), than the n highest-scoring secondary segments are selected. However, if less than n secondary segments within a given group satisfy the threshold snore score, then only those secondary segments satisfying the threshold snore score are selected. If no secondary segments within a given group satisfy the threshold snore score, then no secondary segments from that group are selected. Any suitable threshold snore score can be used. For example, the threshold snore score could be 0.4, 0.5, 0.6, etc.

In some implementations, steps 520, 530, and 540 (and sub-steps 542, 544, 546, and 548) are all performed in real-time during the sleep session. As audio data associated with the sleep session is generated, the audio data corresponding to each respective primary segment is analyzed to determine whether the individual snored during the respective primary segment (e.g., to determine the probability that one or more snoring sounds were made during the respective primary segment). As the primary segments are continually analyzed, the snore scores for each secondary segment can be determined as soon as all of the primary segments contained within a given secondary segment are analyzed. Thus, as soon as the snore probabilities for all of the primary segments within a respective secondary segment are determined, the snore score for that secondary segment can be determined. Further, as the snore scores for the secondary segments are continually determined, the selection of the secondary segments within each group of secondary segments can be performed. Thus, as soon as the snore scores for all of the secondary segments within a respective group of secondary segments are determined, certain of those secondary segments can be selected, and human-perceivable audio samples of the selected secondary segments can be generated.

In some implementations, audio data from the sleep session can be deleted in real-time after it has been used, such as used to complete steps 510-540 (optionally including sub-steps 542-548) as described herein. For example, method 500 can often be implemented on a user device of the individual, such as a smartphone. The audio data from the sleep session can occupy a large amount of space within the memory of the user device, and deleting audio data that is no longer needed can aid in saving space within the memory. For example, the audio data can be generated and analyzed to determine the snore probabilities for the primary segments, and the snore scores for the secondary segments. After a given group of secondary segments is analyzed and audio samples are generated for selected secondary segments within that group, the audio data corresponding to the non-selected secondary segments can be deleted. However, in some implementations, all of the audio data from a sleep session can be retained, on the user device and/or on another device.

Non-audio data associated with the sleep session is generally retained on the user device however. This non-audio data includes data that is indicative of the snore probabilities for the primary segments and the snore scores for the secondary segments. As this non-audio data generally does not occupy as much space as the audio data, it can be retained on the user device and/or on another device. However, in some implementations, the non-audio data can also be deleted.

In some implementations, further analysis of the sleep session can be performed. FIG. 6B shows an example sleep session 650 that includes a plurality of primary segments 660A-660I and a plurality of tertiary segments 670A, 670B, and 670C. Each of the tertiary segments includes multiple primary segments (similar to the secondary segments). Tertiary segment 670A includes primary segments 660A, 660B, and 660C. Tertiary segment 670B includes primary segments 660D, 660E, and 660F. Tertiary segment 670C includes primary segments 660G, 660H, and 660I. While the sleep session 650 shows three tertiary segments 670A-670C and that each tertiary segment contains three primary segments, the primary segments of a sleep session can be divided into the tertiary segments in any suitable manner. Moreover, while the sleep session 600 of FIG. 6A is not shown as having any tertiary segments, and the sleep session 650 of FIG. 6B is not shown as having any secondary segments, the primary segments of a given sleep session can be sorted into one or more secondary segments and one or more tertiary segment. Thus, within a sleep session, each primary segment will generally belong to at least one secondary segment and at least one tertiary segment.

In some implementations, non-audio data from the tertiary segments can be analyzed after the sleep session has ended (or at least after the non-audio data is generated) to provide the individual with additional information related to the sleep session. In these implementations, method 500 can include optional steps 550, 560, and 570, as shown in FIG. 5. At step 550, the sleep session is divided into a plurality to tertiary segments. In some implementations, each of the tertiary segments has a duration of about one minute. In implementations where each of the primary segments has a duration of about one second, each tertiary segment will contain sixty primary segments.

Step 560 includes determining the probability that a snoring sound occurred during each primary segment contained within a respective tertiary segment. In some implementations, these probabilities will have already been determined at step 520. In other implementations however, step 520 includes determining whether a snoring sound occurred in ways other than calculating the probability that a snoring sound occurred, and thus step 560 can include determining these probabilities.

Finally at step 570, a respective tertiary segment of the sleep session can be marked as a snoring segment if the tertiary segment satisfies a predetermined snoring threshold. Tertiary segments that do not satisfy the snoring threshold can be marked as non-snoring segments, or could remain unmarked. A respective tertiary segment satisfies the snoring threshold if the snore probabilities of a threshold number of the primary segments contained within the respective tertiary segment satisfy a threshold snore probability. Thus, if the snore probabilities of a predetermined number of the primary segments contained within the respective tertiary segment satisfy a threshold, the respective tertiary segment is marked as a snoring segment. If a number of primary segments have snore probabilities that satisfy the threshold snore probability, but this number of primary segments is less than the threshold number of primary segments, the respective tertiary segment is not marked as a snoring segment, or is marked as a non-snoring segment.

In some implementations, the threshold snore probability is about 70%. Thus, a threshold number of the primary segments contained within the respective tertiary segment have a snore probability of 70% or higher, then the respective tertiary segment is marked as a snoring segment. In some implementations, the threshold number of primary segments has a total duration that is equal to about 25% of the duration of the respective tertiary segment. For example, in implementations where each tertiary segment contains 60 primary segments (such as implementations where each primary segment has a duration of one second and each tertiary segment has a duration of 60 seconds), the threshold number of primary segments could be fifteen primary segments (e.g., fifteen seconds).

The process in steps 550-570 can be repeated for each tertiary segment of the sleep session, so that any tertiary segment of the sleep session that satisfies the threshold snore probabilities is marked as a snoring segment. This information can be provided to the individual, for example on a display of the user device (e.g., the display device 262 of the user device 260). In some implementations, this information is presented to the individual in the form of a plot displayed on a display of the user device. This plot can show various characteristics of the sleep session, including during which tertiary segments the individual was snoring, the power (or volume, loudness, etc.) of the individual's snoring during the snoring segments, the power (or volume, loudness, etc.) of other sounds during the snoring segments and during non-snoring segments, etc. The plot may also indicate which secondary segments of the sleep session are associated with audio samples that can be selected and listened to by the individual. Additional information associated with the sleep session can also be displayed on the user device of the individual, including the duration of the sleep session (e.g., the amount of time that audio data was generated), the amount of time within the sleep session when the individual was asleep, the percentage of the sleep session during which the individual was asleep, the amount of time within the sleep session during which the individual was snoring, the percentage of the sleep session during which the individual was snoring, the percentage of the sleeping time during the sleep session during which the individual was snoring, etc.

In some implementations, information about the individual's snoring can be displayed relative to (e.g., next to, overlaid on, etc.) a timeline of the individual's sleep session (e.g., a hypnogram), and/or relative to an indication of the individual's breathing signal (e.g., a plot of an audio signal that is representative of the individuals' breathing). Displaying the snoring information in this manner allows the individual to easily view when they snored during their sleep session (e.g., relative to the length of the sleep session, an absolute time of day, during certain sleep stages, etc.). The information can include the total and/or relative snore time, the power (or volume, loudness, etc.) of individual snoring sounds, which portions of the sleep session are associated with audio samples of high-scoring secondary segments, etc. The snore information of the individual could also be displayed relative to a timeline of a bed partner's sleep session (e.g., a timeline that includes information about the bed partner's sleep session such as sleep quality), which would allow the individual and their bed partner to determine if there is any correlation between the individual's snoring and the bed partner's sleep quality. For example, displaying the information in this manner could indicate that the individual's snoring coincides with the bed partner waking up, experiencing micro-awakenings, exiting a REM sleep stage, etc. Further, the power of the snoring sounds could also be correlated with disruptions to the bed partner's sleep session, to further quantify how the individual's snoring impacts the individual's bed partner. The information about the individual's snoring can thus be used to try and mitigate interruptions to the bed partner's sleep quality, such as via the individual using a respiratory therapy device, using a mandibular repositioning device, etc.

As discussed herein, a sleep session can be divided into any number of primary segments, secondary segments, and tertiary segments. Various different segments can also be formed into different groups of segments. As used herein, a primary segment is the type of segment having the shortest duration, and is the type of segment for which the snore probabilities are determined. The snore probabilities for the primary segments are generally determined in real-time during the sleep session, but can in some implementations be determined after the sleep session has finished, or partially in real-time and partially after the sleep session has finished.

Secondary segments have a longer duration than the primary segments, and contain at least two primary segments therein. The secondary segments are the type of segment for which the snore scores are determined (e.g., a snore score based on the power of detected sounds in the secondary segment and the probability that one or more snoring sounds occurred during the secondary segment). The snore scores for the secondary segments are generally determined in real-time during the sleep session, but can in some implementations be determined after the sleep session has finished, or partially in real-time and partially after the sleep session has finished. The secondary segments are also the type of segment for which a human-perceivable audio sample is generated. The secondary segments of the sleep session can be divided into groups of secondary segments, and audio sample of one or more secondary segments can be generated for some or all of the groups of secondary segments. The generation of the audio sample for the secondary segments is generally performed in real-time during the sleep session, but can in some implementations be determined after the sleep session has finished, or partially in real-time and partially after the sleep session has finished.

The tertiary segments also have a longer duration than the primary segments, and contain at least two primary segments therein. The tertiary segments are the type of segment for which it is determined whether the tertiary segment is a snoring segment, based on the snore probabilities of the primary segments contained therein. The determination of whether each tertiary segment is a snoring segment is generally performed after the sleep session has finished, but can in some implementations be performed in real-time during the sleep session, or partially in real-time and partially after the sleep session has finished. The secondary segments and the tertiary segments could contain the same number of primary segments, or could contain different numbers of primary segments. For example, in some implementations, each secondary segment contains enough primary segments to have a duration of about 30 seconds, and each tertiary segment contains enough primary segments to have a duration of about 1 minute.

In some implementations, method 500 (and/or any of the various implementations of method 500 described herein) can be implemented using a system such as system 10. The system includes a control system (such as control system 200 of system 10) and a memory (such as memory device 204 of system 10). The control system includes one or more processors (such as processor 202 of control system 200). The memory has stored thereon machine-readable instructions. The control system is coupled to the memory, and method 500 (and/or any of the various implementations of method 500 described herein) can be implemented when the machine-readable instructions in the memory are expected by at least one of the one or more processors of the control system.

Generally, method 500 can be implemented using a system (such as system 10) having a control system (such as control system 200 of system 10) with one or more processors (such as processor 202 of control system 200), and a memory (such as memory device 204 of system 10) storing machine readable instructions. The control system can be coupled to the memory, and method 500 can be implemented when the machine readable instructions are executed by at least one of the processors of the control system. Method 500 can also be implemented using a computer program product (such as a non-transitory computer readable medium) comprising instructions that when executed by a computer, cause the computer to carry out the steps of method 500.

### Alternative Implmentations

Alternative Implementation 1. A method for analyzing sounds made by an individual during a sleep session, the method comprising: receiving audio data associated with the sleep session, the sleep session being divided into at least a plurality of primary segments; determining, based at least in part on the audio data, whether one or more snoring sounds were made by the individual during each of at least two of the plurality of primary segments; determining a snore score for each of a plurality of secondary segments of the sleep session, each of the plurality of secondary segments containing two or more of the plurality of primary segments; and generating, based at least in part on the snore score for each of the plurality of secondary segments, a human-perceivable audio sample of at least one of the plurality of secondary segments.
Alternative Implementation 2. The method of Alternative Implementation 1, wherein determining whether one or more snoring sounds were made by the individual during each respective primary segment of the at least two primary segments includes determining a probability that at least one snoring sound occurred during the respective primary segment.
Alternative Implementation 3. The method of Alternative Implementation 2, wherein the snore score for each respective secondary segment is based at least in part on the probability that at least one snoring sound occurred during the respective secondary segment.
Alternative Implementation 4. The method of Alternative Implementation 2 or Alternative Implementation 3, wherein the snore score for each respective secondary segment is based at least in part on an average of the probability that at least one snoring sound occurred during each of the two or more primary segments contained within the respective secondary segment.
Alternative Implementation 5. The method of Alternative Implementation 3 or Alternative Implementation 4, wherein the snore score for each respective secondary segment is based at least in part on a power of at least one acoustic signal representing any sounds detected in the respective secondary segment.
Alternative Implementation 6. The method of Alternative Implementation 5, wherein the at least one acoustic signal includes a plurality of acoustic signals each representing any sounds detected in a respective primary segment of the two or more primary segments contained with the respective secondary segment.
Alternative Implementation 7. The method of Alternative Implementation 6, wherein the snore score for each respective secondary segment is based at least in part on a root mean square (RMS) power value of each of the plurality of acoustic signals.
Alternative Implementation 8. The method of Alternative Implementation 7, wherein the snore score for each respective secondary segment is based at least in part on an average of the RMS power value of each of the plurality of acoustic signals.
Alternative Implementation 9. The method of any one of Alternative Implementations 5 to 8, wherein the power of the at least one acoustic signal correlates with a volume of any sounds represented by the at least one acoustic signal.
Alternative Implementation 10. The method of any one of Alternative Implementations 3 to 9, wherein the snore score for each respective secondary segment is based on a weighted sum of (i) the probability that at least one snoring sound occurred during the respective secondary segment, and (ii) the volume of any sounds detected in the respective secondary segment.
Alternative Implementation 11. The method of Alternative Implementation 10, wherein the snore score for each respective secondary segment is based on (i) an average of the probability that at least one snoring sound occurred during each of the two or more primary segments contained within the respective secondary segment, and (ii) an average of an RMS power value of each of two or more acoustic signals, each acoustic signal representing any sounds detected in a respective primary segment of the two or more primary segments contained within the respective secondary segment.
Alternative Implementation 12. The method of Alternative Implementation 10 or Alternative Implementation 11, wherein the snore score for each respective secondary segment is determined according to (0.5*Power_{RMS}+\tilde{P}ₛₙₒᵣₑ)/1.5, where Power_{RMS} is the average RMS power value of acoustic signal representing any sounds detected in each of the two or more primary segments contained within the respective secondary segment, and Psnore is the average of the probability that at least one snoring sound occurred during each of the two or more primary segments contained within the respective secondary segment.
Alternative Implementation 13. The method of any one of Alternative Implementations 1 to 12, wherein generating the human-perceivable audio sample of least one of the plurality of secondary segments includes: comparing the snore score of at least two of the plurality of secondary segments; selecting a set of n highest-scoring secondary segments from the at least two of the plurality of secondary segments; and generating the human-perceivable audio sample of each secondary segment within the selected set of n highest-scoring secondary segments.
Alternative Implementation 14. The method of any one of Alternative Implementations 1 to 12, wherein generating the human-perceivable audio sample of least one of the plurality of secondary segments includes: dividing the plurality of secondary segments into a plurality of groups, each group containing two or more secondary segments; comparing, for each respective group of secondary segments, the snore score of the two or more secondary segments contained within the respective group of secondary segments; selecting, for each respective group of secondary segments, a set of n highest-scoring secondary segments from the two or more secondary segments contained within the respective group of secondary segments; and generating, for each respective group of secondary segments, the human-perceivable audio sample of each secondary segment in the set of n highest-scoring secondary segments contained within the respective group of secondary segments.
Alternative Implementation 15. The method of Alternative Implementation 13 or Alternative Implementation 14, wherein the snore score of each secondary segment within the selected set of n highest-scoring secondary segments satisfies a threshold snore score.
Alternative Implementation 16. The method of Alternative Implementation 15, wherein the snore score of each secondary segment within the set of n highest-scoring secondary segments is greater than or equal to the threshold snore score.
Alternative Implementation 17. The method of any one of Alternative Implementations 13 to 16, wherein n is an integer value that is greater than or equal to one.
Alternative Implementation 18. The method of Alternative Implementation 17, wherein the integer value of n is less than or equal to five.
Alternative Implementation 19. The method of any one of Alternative Implementations 1 to 18, wherein each of the plurality of primary segments of the sleep session has a duration of about one second.
Alternative Implementation 20. The method of Alternative Implementation 19, wherein each of the plurality of secondary segments of the sleep session has a duration of at least about two seconds.
Alternative Implementation 21. The method of Alternative Implementation 20, wherein each of the plurality of secondary segments of the sleep session has a duration of about thirty seconds.
Alternative Implementation 22. The method of Alternative Implementation 20 or Alternative Implementation 21, wherein a sum of the duration of each of the secondary segments in each respective group of two or more secondary segments is greater than or equal to one minute.
Alternative Implementation 23. The method of Alternative Implementation 22, wherein the sum of the duration of each of the secondary segments in each respective group of two or more secondary segments is greater than or equal to one hour.
Alternative Implementation 24. The method of any one of Alternative Implementations 1 to 23, further comprising: dividing the sleep session into a plurality of tertiary segments, each of the plurality of tertiary segments containing two or more of the plurality of primary segments; determining, for each respective one of the plurality of tertiary segments, a probability that at least one snoring sound occurred during each primary segment contained within the respective tertiary segment; and in response to the probability of each of at least a threshold number of primary segments contained within the respective tertiary segment satisfying a threshold probability value, marking the respective tertiary segment as a snoring segment.
Alternative Implementation 25. The method of Alternative Implementation 24, further comprising, in response to the probability for each respective tertiary segment not satisfying the threshold probability value for each of the threshold number of primary segments contained within the respective tertiary segment, marking the respective tertiary segment as a non-snoring segment.
Alternative Implementation 26. The method of Alternative Implementation 24 or Alternative Implementation 25, where the threshold probability value is 70%.
Alternative Implementation 27. The method of any one of Alternative Implementations 24 to 26, wherein the threshold number of primary segments is fifteen primary segments.
Alternative Implementation 28. The method of any one of Alternative Implementations 24 to 27, wherein the threshold number of primary segments has a total duration that is equal to about 25% of a duration of the respective tertiary segment.
Alternative Implementation 29. The method of any one of Alternative Implementations 24 to 28, wherein each respective tertiary segment has a duration of about sixty seconds, and wherein the threshold number of primary segments has a total duration of about fifteen seconds.
Alternative Implementation 30. The method of Alternative Implementation 29, wherein each of the plurality of primary segments has a duration of about one second.
Alternative Implementation 31. The method of any one of Alternative Implementations 24 to 30, wherein dividing the sleep session into the plurality of tertiary segments and determining the probability that at least one snoring sound occurred during each primary segment contained within the respective tertiary segment is performed after the sleep session has ended.
Alternative Implementation 32. The method of any one of Alternative Implementations 1 to 31, wherein determining whether one or more snoring sounds were made by the individual during at least two of the plurality of primary segments is performed in real-time during the sleep session.
Alternative Implementation 33. The method of any one of Alternative Implementations 1 to 32, wherein generating the human-perceivable audio sample of at least one of the plurality of secondary segments is performed in real-time during the sleep session.
Alternative Implementation 34. The method of any one of Alternative Implementations 1 to 33, wherein determining whether one or more snoring sounds were made by the individual during at least two of the plurality of primary segments includes filtering out portions of the audio data corresponding to sounds having a frequency above an upper threshold frequency, sounds having a frequency below a lower threshold frequency, or both.
Alternative Implementation 35. The method of Alternative Implementation 34, wherein the upper threshold frequency is about 5 kHz.
Alternative Implementation 36. The method of Alternative Implementation 34 or Alternative Implementation 35, wherein the lower threshold frequency is about 200 Hz.
Alternative Implementation 37. A system for analyzing sounds made by an individual during a sleep session, the system comprising: a control system including one or more processors; and a memory having stored thereon machine-readable instructions; wherein the control system is coupled to the memory, and the method of any one of Alternative Implementations 1 to 36 is implemented when the machine-readable instructions in the memory are executed by at least one of the one or more processors of the control system.
Alternative Implementation 38. A system for analyzing sounds made by an individual during a sleep session, the system including a control system having one or more processors configured to implement the method of any one of Alternative Implementations 1 to 36.
Alternative Implementation 39. A computer program product comprising instructions which, when executed by a computer, cause the computer to carry out the method of any one of Alternative Implementations 1 to 36.
Alternative Implementation 40. The computer program product of Alternative Implementation 39 wherein the computer program product is a non-transitory computer readable medium.
Alternative Implementation 41. A system for analyzing sounds made by an individual during a sleep session, the system comprising: an electronic interface configured to receive data; a memory storing machine-readable instructions; and a control system including one or more processors configured to execute the machine-readable instructions to: receive audio data associated with the sleep session, the sleep session being divided into at least a plurality of primary segments; determine, based at least in part on the audio data, whether one or more snoring sounds were made by the individual during each of at least two of the plurality of primary segments; determine a snore score for each of a plurality of secondary segments of the sleep session, each of the plurality of secondary segments containing two or more of the plurality of primary segments; and generate, based at least in part on the snore score for each of the plurality of secondary segments, a human-perceivable audio sample of at least one of the plurality of secondary segments.
Alternative Implementation 42. The system of Alternative Implementation 41, wherein determining whether one or more snoring sounds were made by the individual during each respective primary segment of the at least two primary segments includes determining a probability that at least one snoring sound occurred during the respective primary segment.
Alternative Implementation 43. The system of Alternative Implementation 42, wherein the snore score for each respective secondary segment is based at least in part on the probability that at least one snoring sound occurred during the respective secondary segment.
Alternative Implementation 44. The system of Alternative Implementation 42 or Alternative Implementation 43, wherein the snore score for each respective secondary segment is based at least in part on an average of the probability that at least one snoring sound occurred during each of the two or more primary segments contained within the respective secondary segment.
Alternative Implementation 45. The system of Alternative Implementation 43 or Alternative Implementation 44, wherein the snore score for each respective secondary segment is based at least in part on a power of at least one acoustic signal representing any sounds detected in the respective secondary segment.
Alternative Implementation 46. The system of Alternative Implementation 45, wherein the at least one acoustic signal includes a plurality of acoustic signals each representing any sounds detected in a respective primary segment of the two or more primary segments contained with the respective secondary segment.
Alternative Implementation 47. The system of Alternative Implementation 46, wherein the snore score for each respective secondary segment is based at least in part on a root mean square (RMS) power value of each of the plurality of acoustic signals.
Alternative Implementation 48. The system of Alternative Implementation 47, wherein the snore score for each respective secondary segment is based at least in part on an average of the RMS power value of each of the plurality of acoustic signals.
Alternative Implementation 49. The system of any one of Alternative Implementations 45 to 48, wherein the power of the at least one acoustic signal correlates with a volume of any sounds represented by the at least one acoustic signal.
Alternative Implementation 50. The system of any one of Alternative Implementations 43 to 49, wherein the snore score for each respective secondary segment is based on a weighted sum of (i) the probability that at least one snoring sound occurred during the respective secondary segment, and (ii) the volume of any sounds detected in the respective secondary segment.
Alternative Implementation 51. The system of Alternative Implementation 50, wherein the snore score for each respective secondary segment is based on (i) an average of the probability that at least one snoring sound occurred during each of the two or more primary segments contained within the respective secondary segment, and (ii) an average of an RMS power value of each of two or more acoustic signals, each acoustic signal representing any sounds detected in a respective primary segment of the two or more primary segments contained within the respective secondary segment.
Alternative Implementation 52. The system of Alternative Implementation 50 or Alternative Implementation 51, wherein the snore score for each respective secondary segment is determined according to (0.5*Power_{RMS}+\tilde{P}ₛₙₒᵣₑ)/1.5, where Power_{RMS} is the average RMS power value of acoustic signal representing any sounds detected in each of the two or more primary segments contained within the respective secondary segment, and Psnore is the average of the probability that at least one snoring sound occurred during each of the two or more primary segments contained within the respective secondary segment.
Alternative Implementation 53. The system of any one of Alternative Implementations 41 to 52, wherein generating the human-perceivable audio sample of least one of the plurality of secondary segments includes: comparing the snore score of at least two of the plurality of secondary segments; selecting a set of n highest-scoring secondary segments from the at least two of the plurality of secondary segments; and generating the human-perceivable audio sample of each secondary segment within the selected set of n highest-scoring secondary segments.
Alternative Implementation 54. The system of any one of Alternative Implementations 41 to 52, wherein generating the human-perceivable audio sample of least one of the plurality of secondary segments includes: dividing the plurality of secondary segments into a plurality of groups, each group containing two or more secondary segments; comparing, for each respective group of secondary segments, the snore score of the two or more secondary segments contained within the respective group of secondary segments; selecting, for each respective group of secondary segments, a set of n highest-scoring secondary segments from the two or more secondary segments contained within the respective group of secondary segments; and generating, for each respective group of secondary segments, the human-perceivable audio sample of each secondary segment in the set of n highest-scoring secondary segments contained within the respective group of secondary segments.
Alternative Implementation 55. The system of Alternative Implementation 53 or Alternative Implementation 54, wherein the snore score of each secondary segment within the selected set of n highest-scoring secondary segments satisfies a threshold snore score.
Alternative Implementation 56. The system of Alternative Implementation 55, wherein the snore score of each secondary segment within the set of n highest-scoring secondary segments is greater than or equal to the threshold snore score.
Alternative Implementation 57. The system of any one of Alternative Implementations 53 to 56, wherein n is an integer value that is greater than or equal to one.
Alternative Implementation 58. The system of Alternative Implementation 57, wherein the integer value of n is less than or equal to five.
Alternative Implementation 59. The system of any one of Alternative Implementations 41 to 58, wherein each of the plurality of primary segments of the sleep session has a duration of about one second.
Alternative Implementation 60. The system of Alternative Implementation 59, wherein each of the plurality of secondary segments of the sleep session has a duration of at least about two seconds.
Alternative Implementation 61. The system of Alternative Implementation 60, wherein each of the plurality of secondary segments of the sleep session has a duration of about thirty seconds.
Alternative Implementation 62. The system of Alternative Implementation 60 or Alternative Implementation 61, wherein a sum of the duration of each of the secondary segments in each respective group of two or more secondary segments is greater than or equal to one minute.
Alternative Implementation 63. The system of Alternative Implementation 62, wherein the sum of the duration of each of the secondary segments in each respective group of two or more secondary segments is greater than or equal to one hour.
Alternative Implementation 64. The system of any one of Alternative Implementations 41 to 63, wherein the one or more processors of the control system are further configured to execute the machine-readable instructions to: divide the sleep session into a plurality of tertiary segments, each of the plurality of tertiary segments containing two or more of the plurality of primary segments; determine, for each respective one of the plurality of tertiary segments, a probability that at least one snoring sound occurred during each primary segment contained within the respective tertiary segment; and in response to the probability of each of at least a threshold number of primary segments contained within the respective tertiary segment satisfying a threshold probability value, mark the respective tertiary segment as a snoring segment.
Alternative Implementation 65. The system of Alternative Implementation 64, wherein the one or more processors of the control system are further configured to execute the machine-readable instructions to, in response to the probability for each respective tertiary segment not satisfying the threshold probability value for each of the threshold number of primary segments contained within the respective tertiary segment, mark the respective tertiary segment as a non-snoring segment.
Alternative Implementation 66. The system of Alternative Implementation 64 or Alternative Implementation 65, where the threshold probability value is 70%.
Alternative Implementation 67. The system of any one of Alternative Implementations 64 to 66, wherein the threshold number of primary segments is fifteen primary segments.
Alternative Implementation 68. The system of any one of Alternative Implementations 64 to 67, wherein the threshold number of primary segments has a total duration that is equal to about 25% of a duration of the respective tertiary segment.
Alternative Implementation 69. The system of any one of Alternative Implementations 64 to 68, wherein each respective tertiary segment has a duration of about sixty seconds, and wherein the threshold number of primary segments has a total duration of about fifteen seconds.
Alternative Implementation 70. The system of Alternative Implementation 69, wherein each of the plurality of primary segments has a duration of about one second.
Alternative Implementation 71. The system of any one of Alternative Implementations 64 to 70, wherein dividing the sleep session into the plurality of tertiary segments and determining the probability that at least one snoring sound occurred during each primary segment contained within the respective tertiary segment is performed after the sleep session has ended.
Alternative Implementation 72. The system of any one of Alternative Implementations 41 to 71, wherein determining whether one or more snoring sounds were made by the individual during at least two of the plurality of primary segments is performed in real-time during the sleep session.
Alternative Implementation 73. The system of any one of Alternative Implementations 41 to 72, wherein generating the human-perceivable audio sample of at least one of the plurality of secondary segments is performed in real-time during the sleep session.
Alternative Implementation 74. The system of any one of Alternative Implementations 41 to 73, wherein determining whether one or more snoring sounds were made by the individual during at least two of the plurality of primary segments includes filtering out portions of the audio data corresponding to sounds having a frequency above an upper threshold frequency, sounds having a frequency below a lower threshold frequency, or both.
Alternative Implementation 75. The system of Alternative Implementation 74, wherein the upper threshold frequency is about 5 kHz.
Alternative Implementation 76. The system of Alternative Implementation 74 or Alternative Implementation 75, wherein the lower threshold frequency is about 200 Hz.

One or more elements or aspects or steps, or any portion(s) thereof, from one or more of any of the Alternative Implementations and/or claims herein can be combined with one or more elements or aspects or steps, or any portion(s) thereof, from one or more of any of the other Alternative Implementations and/or claims herein or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method for analyzing sounds made by an individual during a sleep session, the method comprising:
receiving audio data associated with the sleep session, the sleep session being divided into at least a plurality of primary segments;
determining, based at least in part on the audio data, whether one or more snoring sounds were made by the individual during each of at least two of the plurality of primary segments;
determining a snore score for each of a plurality of secondary segments of the sleep session, each of the plurality of secondary segments containing two or more of the plurality of primary segments; and
generating, based at least in part on the snore score for each of the plurality of secondary segments, a human-perceivable audio sample of at least one of the plurality of secondary segments.

2. The method of claim 1, wherein determining whether one or more snoring sounds were made by the individual during each respective primary segment of the at least two primary segments includes determining a probability that at least one snoring sound occurred during the respective primary segment.

3. The method of claim 2, wherein the snore score for each respective secondary segment is based at least in part on the probability that at least one snoring sound occurred during the respective secondary segment, or during each of the two or more primary segments contained within the respective secondary segment.

4. (canceled)

5. The method of claim 3, wherein the snore score for each respective secondary segment is based at least in part on a power of at least one acoustic signal representing any sounds detected in the respective secondary segment.

6. The method of claim 5, wherein the at least one acoustic signal includes a plurality of acoustic signals each representing any sounds detected in a respective primary segment of the two or more primary segments contained with the respective secondary segment, and wherein the snore score for each respective secondary segment is based at least in part on an average of a root mean square (RMS) power value of each of the plurality of acoustic signals.

7-9. (canceled)

10. The method of claim 3, wherein the snore score for each respective secondary segment is based on a weighted sum of (i) the probability that at least one snoring sound occurred during the respective secondary segment, and (ii) the volume of any sounds detected in the respective secondary segment.

11. The method of claim 10, wherein the snore score for each respective secondary segment is based on (i) an average of the probability that at least one snoring sound occurred during each of the two or more primary segments contained within the respective secondary segment, and (ii) an average of an RMS power value of each of two or more acoustic signals, each acoustic signal representing any sounds detected in a respective primary segment of the two or more primary segments contained within the respective secondary segment.

12. The method of claim 11, wherein the snore score for each respective secondary segment is determined according to
$$\frac{0.5 * {Power}_{R M S} + {\overline{P}}_{s n o r e}}{1.5} ,$$
where Power_{RMS} is the average of the RMS power value of each of the two or more acoustic signals, and \tilde{P}ₛₙₒᵣₑ is the average of the probability that at least one snoring sound occurred during each of the two or more primary segments contained within the respective secondary segment.

13. The method of claim 1, wherein generating the human-perceivable audio sample of least one of the plurality of secondary segments includes:
comparing the snore score of at least two of the plurality of secondary segments;
selecting a set of n highest-scoring secondary segments from the at least two of the plurality of secondary segments; and
generating the human-perceivable audio sample of each secondary segment within the selected set of n highest-scoring secondary segments.

14. The method of claim 13, further comprising dividing the plurality of secondary segments into a plurality of groups, each group containing two or more secondary segments, and wherein:
comparing the snore score includes comparing, for each respective group of secondary segments, the snore score of the two or more secondary segments contained within the respective group of secondary segments;
selecting the set of n highest-scoring secondary segments includes selecting, for each respective group of secondary segments, a set of n highest-scoring secondary segments from the two or more secondary segments contained within the respective group of secondary segments; and
generating the human-perceivable audio sample includes generating, for each respective group of secondary segments, the human-perceivable audio sample of each secondary segment in the set of n highest-scoring secondary segments contained within the respective group of secondary segments.

15. (canceled)

16. The method of claim 13, wherein the snore score of each secondary segment within the set of n highest-scoring secondary segments is greater than or equal to the threshold snore score.

17-18. (canceled)

19. The method of claim 1, wherein each of the plurality of primary segments of the sleep session has a duration of about one second, about two seconds, or about thirty seconds.

20-21. (canceled)

22. The method of claim 14, wherein a sum of the duration of each of the secondary segments in each respective group of two or more secondary segments is greater than or equal to (i) one minute or (ii) one hour.

23. (canceled)

24. The method of claim 1, further comprising:
dividing the sleep session into a plurality of tertiary segments, each of the plurality of tertiary segments containing two or more of the plurality of primary segments;
determining, for each respective one of the plurality of tertiary segments, a probability that at least one snoring sound occurred during each primary segment contained within the respective tertiary segment; and
in response to the probability of each of at least a threshold number of primary segments contained within the respective tertiary segment satisfying a threshold probability value, marking the respective tertiary segment as a snoring segment.

25. The method of claim 24, further comprising, in response to the probability for each respective tertiary segment not satisfying the threshold probability value for each of the threshold number of primary segments contained within the respective tertiary segment, marking the respective tertiary segment as a non-snoring segment, and wherein the threshold probability value is 70%, the threshold number of primary segments is fifteen primary segments, the threshold number of primary segments has a total duration that is equal to about 25% of a duration of the respective tertiary segment, or any combination thereof.

26-28. (canceled)

29. The method of claim 24, wherein each respective tertiary segment has a duration of about sixty seconds, and wherein the threshold number of primary segments has a total duration of about fifteen seconds.

30-31. (canceled)

32. The method of claim 1, wherein:
(i) determining whether one or more snoring sounds were made by the individual during at least two of the plurality of primary segments is performed in real-time during the sleep session;
(ii) generating the human-perceivable audio sample of the at least one of the plurality of secondary segments is performed in real-time during the sleep session; or
(iii) both (i) and (ii).

33. (canceled)

34. The method of claim 1, wherein determining whether one or more snoring sounds were made by the individual during at least two of the plurality of primary segments includes filtering out portions of the audio data corresponding to sounds having a frequency above an upper threshold frequency, sounds having a frequency below a lower threshold frequency, or both, wherein the upper threshold frequency is about 5 kHz, and wherein the lower threshold frequency is about 200 Hz.

37. A system for analyzing sounds made by an individual during a sleep session, the system comprising:
a control system including one or more processors; and
a memory having stored thereon machine-readable instructions;
wherein the control system is coupled to the memory, and the method of claim 1, is implemented when the machine-readable instructions in the memory are executed by at least one of the one or more processors of the control system.

38. (canceled)

39. A computer program product embodied on a non-transitory computer-readable medium and comprising instructions which, when executed by a computer, cause the computer to carry out the method of claim 1,

40. (canceled)
